(19) United States
(12) Patent Application Publication
(10) Pub. No.: US 2026/0015643 A1
(43) Pub. Date: Jan. 15, 2026

(54) METHOD FOR PRODUCING GLYCOPROTEIN COMPOSITIONS

(71) Applicant: Hoffmann-La Roche Inc., Little Falls, NJ (US)

(72) Inventors: Marina Luise BOYD, Geretsried (DE); Christian KLINGER, Penzberg (DE)

(73) Assignee: Hoffmann-La Roche Inc., Little Falls, NJ (US)

(21) Appl. No.: 19/195,365

(22) Filed: Apr. 30, 2025

Related U.S. Application Data
(63) Continuation of application No. PCT/EP2023/079453, filed on Oct. 23, 2023.

(30) Foreign Application Priority Data
Nov. 2, 2022 (EP) 22205031.2

(51) Int. Cl. C12P 21/00; C07K 14/505

(52) U.S. Cl. CPC C12P 21/005; C07K 14/505

(57) ABSTRACT

The present invention relates to a method for recombinantly producing a glycoprotein composition comprising at least one glycoprotein comprising the step of cultivating a recombinant host cell which expresses the glycoprotein in a cultivation medium in a bioreactor having a pH measuring device positioned in the bioreactor and configured to be in physical contact with the cultivation medium, characterized in that (a) the cultivating is performed under sterile conditions, (b) the pH value measured with the pH measuring device differs by 0.05 units or less from the pH value of the cultivation medium, and (c) the relative content of at least one glycosylated variant in the glycoprotein composition has reduced batch-to-batch variability, compared to a method where the pH value measured by the pH measuring device differs by more than 0.05 units, preferably by more than 0.03 units, from the pH value in the cultivation medium, and thereby producing the glycoprotein composition. The present invention further relates to glycoprotein compositions produced by the method, and to the use of a method for carbon dioxide based pH calibration for improving batch-to-batch variability.

### CROSS-REFERENCE TO RELATED APPLICATIONS

This application is a National Stage Entry of International Application No. PCT/EP2023/079453, filed Oct. 23, 2023, which claims benefit of priority to European Patent Application No. 22205031.2, filed Nov. 2, 2022, each of which is incorporated herein by reference in its entirety.

### SEQUENCE LISTING

This application contains a Sequence Listing which has been submitted electronically in XML format and is hereby incorporated by reference in its entirety. Said XML copy, created on Apr. 28, 2025, is named P37874-US_SL.xml and is 2,030 bytes in size.

### TECHNICAL FIELD

The present invention relates to methods for recombinantly producing a glycoprotein composition with reduced batch-to-batch variability. The methods disclosed herein include a pH measurement step wherein the measured pH value differs by 0.05 units or less from the pH value of the cultivation medium, leading to reduced batch-to-batch variability in one or more glycosylated variants in a glycoprotein composition, compared to a method where the pH value measured by the pH measuring device differs by 0.05 units or less from the pH value in the cultivation medium.

### BACKGROUND

Recombinant glycoproteins are commonly produced in eukaryotic expression systems, as eukaryotic cells possess the necessary glycosylation machinery, i.e. the enzymes that are required to attach sugar moieties to the protein. Variation of the glycan pattern on proteins has enormous implications on protein function. For example, the structure of the N-linked glycans on a protein has an impact on various characteristics, including the protease susceptibility, intracellular trafficking, secretion, tissue targeting, biological half-life and antigenicity of the protein in a cell or organism. The alteration of one or more of these characteristics greatly affects the efficacy of a glycoprotein in its natural setting, and consequently also may affect the efficacy of a glycoprotein as a therapeutic agent. The glycosylation status of a glycoprotein is tightly regulated and even small differences in glycosylation can have significant effects.

In glycoproteins, sugars are attached either to the amide nitrogen atom in the side chain of asparagine (an N-linkage) or to the oxygen atom in the side chain of serine or threonine (an O-linkage). Glycosylation starts with the formation of the N-linkages in the endoplasmic reticulum, so-called “core glycosylation”. After this, the polypeptides are transported to the Golgi apparatus where the O-linked sugars are added and the N-linked sugar chains are modified in many different ways, for example by the removal of mannose residues, addition of N-acetylglucosamine, galactose and/or fucose and/or sialic acid residues.

Erythropoiesis stimulating glycoproteins comprise several N-glycosylation sites. Erythropoietin (EPO) is a glycoprotein with three N-glycosylation sites and one O-glycosylation site. Erythropoietin has been manufactured biosynthetically using recombinant DNA technology (Egrie, J C, Strickland, T W, Lane, J et al. (1986) Immunobiol. 72:213-224) and is the product of a cloned human EPO gene inserted into and expressed in the ovarian tissue cells of the Chinese hamster (CHO cells). The primary structure of the predominant, fully processed form of human erythropoietin (hEPO) is illustrated in FIG. 1. There are two disulfide bridges: between Cys7-Cys161 and Cys29-Cys33. The molecular weight of the polypeptide chain of erythropoietin without the sugar moieties is 18,236 Da. In the intact erythropoietin glycoprotein, approximately 40% of the molecular weight is accounted for by the carbohydrate groups that glycosylate the protein (Sasaki, H, Bothner, B, Dell, A and Fukuda, M (1987) J. Biol. Chem. 262:12059).

Typical N-Glycans of erythropoietin include bi-, tri- and tetraantennary structures with one or two N-acetyl lactosamine repeats (see e.g. Postnikov et al. 2016 Russ. Chem. Rev. 85-99). It is known that a higher number of terminal sialic acid moieties on N-Glycans of erythropoietin is associated with a higher specific activity of erythropoietin when compared to less sialylated erythropoietin glycoproteins (see e.g. Imai et al., Eur. J. Biochem. 194 (1990), p. 457-462). Desialylation of erythropoietin reduces the half-life of erythropoietin in the circulation (Ridley et al. J Natl Med Assoc. 1994 February; 86(2): 129-35). A higher number of poly-N-acetyl lactosamine repeats or a higher number of branches of the N-Glycans of erythropoietin are both reported to be associated with a higher specific bioactivity of erythropoietin, respectively (see e.g. WO 99/28346). The glycosylated variants of erythropoietin are not just key influencing factors on the biological activity, in vivo bioactivity and pharmacokinetics of erythropoietin, they also alter the solubility, and lifetime of glycoproteins in the blood.

In the ICH Guidelines, a Critical Quality Attribute (CQA) is defined as a physical, chemical, biological, or microbiological property or characteristic that should be within an appropriate limit, range, or distribution to ensure the desired product quality (International conference on harmonisation of technical requirements for registration of pharmaceuticals for human use, Pharmaceutical Development, Q8 (R2)). Because of the described potential impact on bioactivity, N-glycosylation of recombinantly produced erythropoietin is considered to be a critical quality attribute (CQA) and is carefully monitored during production to ensure consistent product quality. The relative content in glycosylated variants strongly depends on several factors, including cell culture conditions. Due to the critical influence of glycosylated variants on bioavailability, the ability to produce consistently a narrow range of a glycosylation variant of a pharmaceutical erythropoietin product thus also translates into a lower batch-to-batch variability with regard to erythropoietin specific activity.

Yoon at al. (Biotechnol Bioeng, 89 (3): 345-356, 2005) discloses that optimization of culture temperature and pH in serum-free suspension culture of CHO cells can result in increase of maximum erythropoietin concentration and volumetric productivity.

WO 2017/072340 A1 discloses a system for monitoring deviations of a state of a cell culture in a bioreactor from a reference state of a cell culture in a reference bioreactor, wherein the bioreactor comprises the same medium as the reference bioreactor.

WO 2017/072346 A1 discloses a system and method for determining if a pH measuring device is affected by a pH-measuring problem and for re-calibrating a pH meter, using CO₂ concentrations measured in two or more different tanks for identifying pH-measuring deviations of the pH measuring devices which are operatively coupled to the different tanks.

Thus, there is a continuing interest in methods for producing erythropoietin in reliably high quality.

### SUMMARY

The invention is based on the finding that strictly controlling the pH of a cultivation medium in a production bioreactor during fermentation results in considerably improved variability of a glycoprotein product, and in particular of the N-glycosylation profile of erythropoietin, particularly with regard to the presence of sialylated isoforms of erythropoietin and of tetraantennary+1 repeat structures.

The inventors have found that by adjusting a bioreactor pH probe signal to a pH reference that accurately reflects the pH in a cultivation medium, unaffected by any offsets that may be introduced by sampling and offline measurement, and optionally also by subsequently controlling the pH in the cultivation medium with such a pH probe, it is possible to reduce the variability with regard to N-glycosylation and sialylation during the production of a glycoprotein composition in a recombinant mammalian host cell considerably.

It has been found that using a method for calibrating an in-line pH measuring device after sterilizing and filling a bioreactor with cultivation medium that comprises the steps of
a) determining the carbon dioxide concentration in the bioreactor's headspace and/or exhaust,
b) using a media specific correlation to calculate the pH value of the cultivation medium in the bioreactor, and
c) adjusting the pH measuring device to said pH value,
leads to a decrease in the standard deviation of the relative content of one or more glycosylated variants in a glycoprotein composition produced in such a bioreactor, compared to the standard deviation of the relative content of the same glycosylated variants in glycoprotein compositions which have been produced using a method wherein the pH value measured by the pH measuring device in the bioreactor differs by more than 0.05 units, preferably by more than 0.03 units, from the pH of the cultivation medium, in particular such methods that rely on offline measurement of pH to (re-) calibrate the in-line pH measuring device. The standard deviation of the relative content of the one or more glycosylated variants is thereby calculated for glycoprotein compositions from at least two fermentation batches.

One aspect of the invention is thus a method for recombinantly producing a glycoprotein composition comprising at least one glycoprotein, wherein the method comprises the step of cultivating a recombinant host cell which expresses the glycoprotein in a cultivation medium in a bioreactor having a pH measuring device positioned in the bioreactor and configured to be in physical contact with the cultivation medium,
characterized in that
(a) the cultivating is performed under sterile conditions,
(b) the pH value measured with the pH measuring device differs by less than 0.05 units from the pH value of the cultivation medium, and
(c) the relative content of at least one glycosylated variant in the glycoprotein composition has reduced batch-to-batch variability, compared to a method wherein the pH value measured by the pH measuring device differs by more than 0.05 units, preferably by more than 0.03 units, from the pH value in the cultivation medium,
and thereby producing the glycoprotein composition.

In certain embodiments of all aspects and embodiments, the method wherein the pH value measured by the pH measuring device differs by more than 0.05 units, preferably by more than 0.03 units, from the pH value in the cultivation medium is a method that comprises removing a sample of the cultivation medium from the bioreactor, in particular under sterile conditions, measuring the pH value of the sample with a pH measurement device that is positioned outside the bioreactor (“off-line pH measurement”) and adjusting the pH measurement device in the bioreactor to the measured pH value.

Another aspect is a method for recombinantly producing a glycoprotein composition comprising at least one glycoprotein in a recombinant host cell which expresses the glycoprotein, comprising the steps of
(a) providing a bioreactor comprising a pH measuring device positioned in the bioreactor and configured to be in physical contact with a cultivation medium,
(b) closing and sterilizing the bioreactor,
(c) filling the cultivation medium into the bioreactor,
(d) calibrating the pH measuring device,
(e) inoculating the bioreactor with the recombinant host cell, and
(f) cultivating the recombinant host cell under conditions suitable for producing the glycoprotein, and
(g) thereby producing the glycoprotein,
characterized in that the calibrating of step (d) comprises the steps of
(i) introducing a gas mixture comprising carbon dioxide gas into the bioreactor,
(ii) determining the carbon dioxide concentration in the headspace and/or the exhaust gas of the bioreactor,
(iii) calculating the pH of the cultivation medium based on a media specific correlation, and
(iv) adjusting said pH measuring device to the pH calculated in step (iii).

In certain embodiments of the aspect, the pH measured with the pH measuring device after the calibration step (d) differs by 0.05 units or less, preferably by 0.03 units or less, from the pH value of the cultivation medium.

In certain embodiments of all aspects and embodiments, the glycoprotein composition comprises at least one glycosylated variant of the glycoprotein, and the standard deviation of the relative content of the glycosylated variant calculated for glycoprotein compositions from at least two fermentation batches is decreased, compared to the standard deviation of the relative content of the glycosylated variant calculated for glycoprotein compositions which have been produced using a method wherein the pH value measured by the pH measuring device differs by more than 0.05 units, preferably by more than 0.03 units, from the pH value in the cultivation medium.

In certain embodiments, the difference between the pH value measured with the pH measuring device in the bioreactor and the pH in the cultivation medium is the only parameter that is changed in the production of the fermentation batches when comparing the standard deviation of a glycosylated variant of a glycoprotein composition produced according to the invention to the standard deviation of another glycoprotein composition.

In certain embodiments of all aspects and embodiments, the standard deviation is calculated for glycoprotein compositions from at least three, preferably at least five, more preferably at least ten, fermentation batches.

In certain embodiments of all aspects and embodiments, the recombinant host cell is a mammalian cell, particularly a CHO cell.

In certain embodiments of all aspects and embodiments, the cultivation medium comprises a carbonate buffer.

In certain embodiments of all aspects and embodiments, the media specific correlation has been determined by collecting at least one medium-specific data-set for the cultivation medium, wherein the data-set is collected using the steps of
(a) filling the cultivation medium into a bioreactor, wherein the bioreactor has at least one pH measuring device positioned in the bioreactor and configured to be in physical contact with a cultivation medium,
(b) introducing a gas mixture comprising carbon dioxide into the bioreactor,
(c) measuring the pH in the cultivation medium with the pH measuring device wherein the pH measuring device has been calibrated (under non-sterile conditions) to measure the pH in the cultivation medium,
(d) varying either
   (i) the pH value in the cultivation medium and measuring the carbon dioxide concentrations in the headspace and/or exhaust of the bioreactor for at least two different pH values, or
   (ii) the carbon dioxide concentration in the gas mixture and measuring the pH values for at least two different carbon dioxide concentrations in the gas mixture, and
(e) obtaining the media specific correlation by mathematically fitting at least two pairs of headspace/exhaust carbon dioxide concentration and corresponding pH value of the cultivation medium.

In certain embodiments of all aspects and embodiments, the recombinant host cells have been cultivated, prior to inoculation, in a pre-culture medium in at least one pre-culture step, wherein the bioreactor used for pre-culture has at least one pH measuring device positioned in the bioreactor and configured to be in physical contact with a pre-culture medium, and wherein the pH measuring device in the bioreactor used for pre-culture is calibrated according to the steps of
(i) introducing a gas mixture comprising carbon dioxide gas into the bioreactor
(ii) determining the carbon dioxide concentration in the headspace and/or the exhaust gas,
(iii) calculating the pH value of the pre-culture medium based on a media specific correlation, and
(iv) adjusting said pH measuring device to the pH value calculated in step (iii).

In certain embodiments of all aspects and embodiments, the glycoprotein composition is an erythropoietic composition. In certain embodiments of all aspects and embodiments, the glycoprotein is an erythropoiesis-stimulating glycoprotein. In certain embodiments of all aspects and embodiments, the glycoprotein is erythropoietin.

One aspect of the invention is a method of reducing the variability of the relative content of at least one glycosylated variant between batches of a recombinant glycoprotein composition, the method comprising:
(a) providing a bioreactor comprising a pH measuring device positioned in the bioreactor and configured to be in physical contact with a cultivation medium,
(b) closing and sterilizing the bioreactor,
(c) filling the cultivation medium into the bioreactor,
(d) calibrating the pH measuring device,
(e) inoculating the bioreactor with a recombinant host cell which expresses the glycoprotein,
(f) cultivating the recombinant host cell under conditions suitable for producing the glycoprotein, and
(g) thereby producing the glycoprotein,
wherein the calibrating of step (d) comprises the steps of
   (i) introducing a gas mixture comprising carbon dioxide gas into the bioreactor,
   (ii) determining the carbon dioxide concentration in the headspace and/or the exhaust gas,
   (iii) calculating the pH value of the cultivation medium based on a media specific correlation, and
   (iv) adjusting said pH measuring device to the pH value calculated in step iii), and
wherein steps (a) to (g) result in a first batch of said recombinant glycoprotein composition with a defined relative content of the glycosylated variant,
(h) repeating the steps (a) to (g) resulting in at least one subsequent batch of said glycoprotein composition, wherein the relative content of the at least one glycosylated variant of said first and at least one subsequent batch has reduced batch-to-batch variability.

In certain embodiments of all aspects and embodiments, the standard deviation of the relative content of the glycoprotein calculated for said first and at least one subsequent batch is 1% or less, particularly 0.8% or less, most particularly 0.5% or less, of the median value of the pH value.

One aspect of the invention is the use of a carbon dioxide based method of calibrating a pH measurement device positioned in a bioreactor and configured to be in physical contact with a cultivation medium in the bioreactor for reducing the standard deviation of the relative content of one or more glycosylated variants in a glycoprotein composition calculated between at least two batches, compared to a glycoprotein composition produced using a pH measurement method where the measured pH value differs by more than 0.05 units, preferably by more than 0.03 units, from the pH value in the cultivation medium.

One further aspect of the invention is an erythropoietic composition comprising at least one erythropoiesis-stimulating glycoprotein wherein (a) about 3.3 area-% to about 3.8 area-% of the N-Glycans of the erythropoiesis-stimulating glycoprotein have biantennary structure; (b) about 8.6 area-% to about 9.5 area-% of the N-Glycans of the erythropoiesis-stimulating glycoprotein have triantennary structure; (c) about 5.6 area-% to about 5.9 area-% of the N-Glycans of the erythropoiesis-stimulating glycoprotein have triantennary+1 repeat structure; (d) about 42.2 area-% to about 43.4 area-% of the N-Glycans of the erythropoiesis-stimulating glycoprotein have tetraantennary structure; (e) about 27.4 area-% to about 28.1 area-% of the N-Glycans of the erythropoiesis-stimulating glycoprotein have tetraantennary+1 repeat structure; (f) about 10.7 area-% to about 11.6 area-% of the N-Glycans of the erythropoiesis-stimulating glycoprotein have tetraantennary+2 repeat structure, (g) about 13.2 area-% to about 16.0 area-% of the erythropoiesis-stimulating glycoprotein are of Isoform 2; (h) about 24.1 area-% to about 26.5 area-% of the erythropoiesis-stimulating glycoprotein are of Isoform 3; (i) about 23.5 area-% to about 24.6 area-% of the erythropoiesis-stimulating glycoprotein are of Isoform 4; (j) about 17.1 area-% to about 18.6 area-% of the erythropoiesis-stimulating glycoprotein are of Isoform 5; (k) about 9.4 area-% to about 11.8 area-% of the erythropoiesis-stimulating glycoprotein are of Isoform 6; (1) about 3.7 area-% to about 5.5 area-% of the erythropoiesis-stimulating glycoprotein are of Isoform 7; and/or (m) about 0.9 area-% to about 1.6 area-% of the erythropoiesis-stimulating glycoprotein are of Isoform 8. In certain embodiments of all aspects and embodiments, the area-percent of N-Glycans of the erythropoiesis-stimulating glycoprotein are determined by anion exchange chromatography after enzymatic release of N-glycosidically-linked oligosaccharides from the glycoprotein and (enzymatic) desialylation. In certain embodiments, the anion exchange chromatography is a high performance anion-exchange chromatography with pulsed amperometric detection (HPAEC-PAD). In certain embodiments of all aspects and embodiments, the area-percent of the sialylated isoforms of the erythropoiesis-stimulating glycoprotein in the erythropoietic composition are determined by capillary zone electrophoresis.

The invention provides for a method for generating glycoproteins, in particular for erythropoiesis stimulating glycoproteins, e.g. erythropoietin, with a reduced batch-to-batch variability with regard to glycosylation, particularly with regard to N-glycans with tetraantennary, tetraantennary+1 repeat, tetraantennary+2 repeats and tetraantennary+3 repeats structure, and to sialylation isoforms, in particular, isoforms 2 and 3, which may assure a constant product quality and/or improve the biological function, such as the specific bioactivity, of the (erythropoiesis stimulating) glycoprotein.

### Description of the Amino Acid Sequences

| | |
|---|---|
| SEQ ID | Human erythropoietin 165 aa |
| NO: 1 | APPRLICDSR VLERYLLEAK EAENITTGCA EHCSLNENIT VPDTKVNFYA |
| | WKRMEVGQQA VEVWQGLALL SEAVLRGQAL LVNSSQPWEP LQLHVDKAVS |
| | GLRSLTTLLR ALGAQKEAIS PPDAASAAPL RTITADTFRK LFRVYSNFLR |
| | GKLKLYTGEA CRTGD |

### BRIEF DESCRIPTION OF THE FIGURES

FIG. 1: Structure of erythropoietin and its glycosylation sites (adapted from Postnikov et al 2016 Russ. Chem. Rev. 85-99)

FIG. 2: Typical glycosylation patterns of erythropoietin.

FIG. 2A: N-Glycan with two branches.

FIG. 2B: N-Glycan with three branches.

FIG. 2C: N-Glycan with four branches.

FIG. 2D: N-Glycan with four branches and one poly N-acetyl lactosamine repeat.

FIG. 2E: N-Glycan with four branches and three poly N-acetyl lactosamine repeats.

FIG. 3: A correlation of bioreactor pH and corresponding exhaust carbon dioxide concentration (“ACO”) manufactured independently at three different pressures, 20 mbar (top), 50 mbar (middle) and 135 mbar (bottom). The lines represent quadratic fits.

FIGS. 4A & 4B: Improved range and standard deviation for erythropoietin glycosylated variants of isoform 2.

FIGS. 5A & 5B: Improved range and standard deviation for erythropoietin glycosylated variants with tetraantennary+1 repeat N-glycan structure.

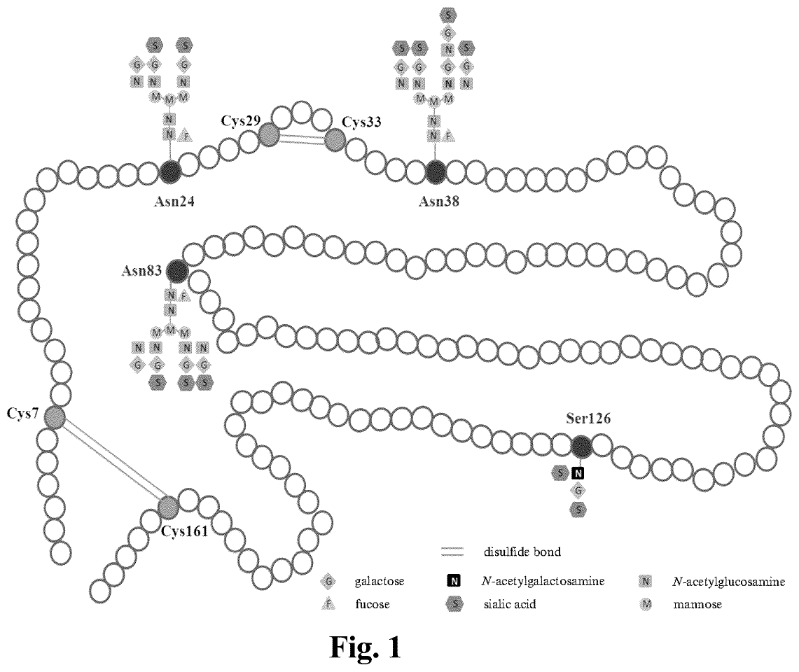

### DETAILED DESCRIPTION

### 1. Definitions

In order that the present invention may be more readily understood, certain terms are first defined. Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless otherwise defined herein, scientific and technical terms used in connection with the present invention shall have the meanings commonly ascribed to them in the art. The meaning and scope of the terms should be clear, however, and in the event of any latent ambiguity, definitions provided herein take precedent over dictionary or extrinsic definition. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “about” denotes a range of +/−20% of the numerical value following thereafter. In certain embodiments, the term “about” denotes a range of +/−10% of the numerical value following thereafter. In certain embodiments, the term “about” denotes a range of +/−5% of the numerical value following thereafter.

The term “comprising” also encompasses the term “consisting of”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The term “accuracy”, as used herein refers to the closeness of a reported or estimated value from the true value. An inaccurate measurement, observation, or estimation differs from the true value. An accurate measurement, observation, or estimation does not differ from the true value.

The terms “aerating”, “aeration” or “gassing” are used herein synonymously and refer to the dispersion of a gaseous material into a flowable fluid, such as a cultivation medium, for example to provide a diffusion surface to introduce a molecule or compound from the gas into the flowable surface. The term is not limited to the dispersion of air per se, but rather refers to the introduction of any gas to a cultivation medium, e.g. oxygen, carbon dioxide, nitrogen, and the like and mixtures thereof. In certain embodiments of all aspects and embodiments, a cell culture is aerated with a defined gas mixture, e.g. comprising process air and carbon dioxide. For normal cell growth, a certain concentration of dissolved oxygen must be maintained. Also, controlled introduction of carbon dioxide is used to maintain pH at desired levels. In certain embodiments, the gas mixture introduced into a bioreactor during calibrating the pH measuring device and/or during cultivating comprises 93% process air and 7% CO₂. Aeration may be performed by head-space aeration, although for large bioreactors the gas-exchange achieved by head-space aeration may be too slow. The most efficient way of introducing gases into bioreactor fluid is submerse gassing or sparging (whereby these two terms are used synonymously herein), which involves forming small bubbles in the fluid. The aeration of the cultivation medium by sparging is preferred to allow molecules and/or compounds to diffuse to the cultivation medium through the fluid-bubble interface following expulsion of the bubbles into the cultivation medium. In one embodiment, aeration is achieved by sparging the cultivation medium with a gas with a defined carbon dioxide concentration. In certain embodiments, the cultivation medium is sparged with a gas mixture that comprises 93% process air and 7% CO₂. In some embodiments, porous solid materials (like titanium) or metal sparging rings with small pre-drilled holes associated with the bioreactor provide sparging.

As used herein, the term “area-percent” or “area-%” refers to the percentage of a particular species' (for example, of a certain glycosylated variant of a glycoprotein) integrated area under the peak (hereinafter “AUP”), as measured by a detector, relative to the total integrated peak area of the entire chromatogram, for example of a chromatogram generated by high performance anion-exchange chromatography with pulsed amperometric detection or by capillary zone electrophoresis. AUP can be determined by using a suitable integrator. Each peak in the chromatogram corresponds to a different component in the mixture which was loaded onto the chromatographic column, and the ratio of the AUP of each of the detectable components with the total AUPs of all the sample components results in the area percentage. Area percent can be expressed mathematically as:

$${area}_{i} - \% = 100 \times ( {AUP}_{i} ) / ( \sum of all AUPs )$$

The term “bioreactor” as used herein refers to any biocompatible tank or vessel, such as a large fermentation chamber, used for the growth of a mammalian cell culture. Typically a bioreactor will be at least 0.25 L and may be 1, 10, 100, 250, 500, 1000, 2500, 5000, 8000, 10,000, 12,000 L or more, or any volume in between. In one embodiment of all aspects and embodiments herein, the bioreactor is a large-scale bioreactor, that is, the bioreactor will be at least 10 L, preferably at least 50 L, more preferably at least 500 L, particularly preferably at least 5000 L, and/or is used for recombinant glycoprotein production for commercial purposes. Typically, the internal conditions of the bioreactor, including but not limited to pH, dissolved oxygen, agitation, temperature and/or pH, can be monitored, adjusted and controlled during the cultivation. When cultivation medium has been filled into the bioreactor, the volume of the bioreactor interior is typically not completely filled, but is divided in a working volume and a headspace volume, wherein the working volume is the fraction of the total volume taken up by the cultivation medium, while the remaining volume above the cultivation medium is referred to herein as “headspace” or “headspace volume”. Typically, the working volume will be about 70% to 80% of the total bioreactor volume. The bioreactor includes at least one inlet and one outlet that allows for introducing and removing gas streams, respectively. These may comprise air inlet filters that provide for sterile gas mixtures for aseptic conditions within the bioreactor. The gas stream that leaves the bioreactor through the outlet is also referred to herein as “exhaust”. The outlet may comprise sensors that allow for the determination of the carbon dioxide concentration in the exhaust. Such sensors may be, e.g. infrared-based off-gas analyzers, mass spectrometry, RAMAN sensors, or optical devices. Bioreactors are further typically in fluid communication with other vessels, for instance a cultivation medium tank or a harvest tank, to allow for introducing or removing liquid media to and from the bioreactor. It will be apparent to those skilled in the art that one or more additional access ports may be provided for greater access to the interior of the bioreactor, including access under aseptic/sterile conditions, for instance for removal of samples that may be used for offline measurements. The bioreactor may further have additional equipment, for example impellers, baffles, spargers and/or ports, which specifically allows for the cultivation and propagation of mammalian cells. A bioreactor can be composed of any material that is suitable for holding mammalian cell cultures suspended in media under the culture conditions of the present invention, including glass, plastic or metal or a combination thereof. A bioreactor may be multi- or single-use, re-usable, disposable or recyclable.

The term “buffer” as used herein, refers to a substance which, by its presence in solution, increases the amount of acid or alkali that must be added to cause unit change in pH value. A buffered solution resists changes in pH by the action of its acid-base conjugate components. Buffered solutions for use with biological reagents are generally capable of maintaining a constant concentration of hydrogen ions such that the pH of the solution is within a desired range, which is often close to physiological pH. Traditional buffer components include, but are not limited to, organic and inorganic salts, acids and bases. Well-known buffer substances are for instance phosphate buffered solutions consisting of phosphoric acid and/or salts thereof, acetate buffered solutions consisting of acetic acid and salts thereof, carbonate and/or bicarbonate buffered solutions, citrate buffered solutions consisting of citric acid and/or salts thereof, morpholine buffered solutions, 2-(N-morpholino) ethanesulfonic buffered solution, histidine buffered solutions, glycine buffered solutions, a tris (hydroxymethyl) aminomethane (TRIS) buffered solutions.

The terms “carbon dioxide concentration” and “fraction of carbon dioxide (gas)” are used herein interchangeably and refer to the relative amount of carbon dioxide gas present in a gas mix. The carbon dioxide concentration is expressed in [%] herein.

The terms “cell culture” or “cell cultivation” refer to a cell population that is suspended in a cultivation medium under conditions suitable for survival and/or growth of the cell population. The terms will also be applied to the combination of the cultivation medium and cell population suspended therein. The terms include cell cultivation processes, also referred to as “fermentation” or “fermentation process”, in all scales (e.g. from Ambr® systems to large-scale industrial bioreactors, i.e. from milliliter-scale to >10.000 L scale), in all different process modes (e.g. batch, fed-batch, perfusion, continuous cultivation), in all process control modes (e.g. non-controlled, fully automated and controlled systems with control of e.g. pH, temperature, oxygen content), and in all kind of cell cultivation systems (e.g. single-use systems, stainless steel systems, glass ware systems). In general, the following parameters are often determined on a daily basis and cover the viable cell concentration, product concentration and several metabolites such as glucose or lactic acid, pH, osmolarity, osmolality and ammonium. In a preferred embodiment of the present invention, the cell culture is a mammalian cell culture and is a batch or a fed-batch cultivation. Commonly, cell culture is performed under sterile, controlled temperature and atmospheric conditions in adherent culture or in suspension culture. Cultures can be grown in shake flasks, small-scale bioreactors, and/or large-scale bioreactors. In certain embodiments of all aspects and embodiments herein, the methods of the invention may be used in large-scale mammalian cell culture, i.e. in bioreactors with at least 10 L, preferably at least 50 L, more preferably at least 500 L, particularly preferably at least 5000 L, and/or is used for recombinant glycoprotein production for commercial purposes. In one embodiment, the cell culture methods and compositions of the invention are suitable for large-scale CHO cell culture and glycoprotein production. In one embodiment of all aspects and embodiments herein, the recombinant glycoprotein production is for commercial purposes and/or is performed using a large-scale bioreactor.

Once a glycoprotein described herein has been produced by recombinant expression, it can be purified by any method known in the art for purification, for example, by chromatography (e.g., ion exchange, affinity, and size exclusion column chromatography), centrifugation, differential solubility, or by any other standard technique for the purification of proteins. For example, an antibody can be isolated and purified by appropriately selecting and combining affinity columns such as Protein A column with chromatography columns, filtration, ultrafiltration, salting-out and dialysis procedures (see, e.g., Antibodies: A Laboratory Manual, Ed Harlow, David Lane, Cold Spring Harbor Laboratory, 1988). Further, as described herein, a glycoprotein can be fused to heterologous polypeptide sequences to facilitate purification. Polypeptides having desired sugar chains can be separated with a lectin column by methods known in the art.

The term “cultivation medium” or “cultivation media” as used herein means a liquid solution used to provide nutrients (e.g., vitamins, amino acids, essential nutrients, salts, and the like) and properties (e.g., similarity, buffering) to maintain living cells, in particular mammalian cells, and support their growth. Preferably, mammalian cells may be cultured at a neutral pH, such as from about pH 6.5 to about pH 7.5, preferably from about pH 6.6 to about pH 7.3, more preferred at a pH of about 7. Hence, buffering agents should be added to the cultivation medium. Commercially available cultivation media are known to those skilled in the art. In one embodiment of all aspects and embodiments, the cultivation medium is buffered using a carbonate buffer system. A cultivation medium that is used during a pre-culture step is termed “pre-culture medium” herein. It may be of the same or a different composition as the cultivation medium used for main stage cultivation.

The term “glycoprotein” as used herein refers to a protein or polypeptide that contains one or more covalently linked oligosaccharide chains. The oligosaccharide chains may be composed of a single sugar residue, a single unbranched chain of sugar residues or may be composed of a chain of sugar residues that branches one or more times. In certain embodiments, oligosaccharide chains are N-linked. In certain embodiments, oligosaccharide chains are O-linked. The term “glycoprotein composition” as used herein refers to a composition comprising at least one glycoprotein.

Glycoproteins include, for example, any of a variety of hematologic agents (including, for instance, erythropoietin, blood-clotting factors, etc.), interferons, colony stimulating factors, antibodies, enzymes, and hormones. The identity of a particular glycoprotein is not intended to limit the present disclosure, and a therapeutic preparation described herein can include any glycoprotein of interest, e.g., a glycoprotein having an Fc region.

A glycoprotein disclosed herein can include a target-binding domain that binds to a target of interest (e.g., binds to an antigen). For example, a glycoprotein, such as an antibody, can bind to a transmembrane polypeptide (e.g., receptor) or ligand (e.g., a growth factor). Exemplary molecular targets (e.g., antigens) for glycoproteins described herein (e.g., antibodies) include CD proteins such as CD2, CD3, CD4, CD8, CD11, CD19, CD20, CD22, CD25, CD33, CD34, CD40, CD52; members of the ErbB receptor family such as the EGF receptor (EGFR, HER1, ErbB1), HER2 (ErbB2), HER3 (ErbB3) or HER4 (ErbB4) receptor; macrophage receptors such as CRIg; tumor necrosis factors such as TNFa or TRAIL/Apo-2; cell adhesion molecules such as LFA-1, MacI, p150,95, VLA-4, ICAM-1, VCAM and αvβ3 integrin including either a or p subunits thereof (e.g., anti-CD11a, anti-CD18 or anti-CD11b antibodies); growth factors and receptors such as EGF, FGFR (e.g., FGFR3) and VEGF; IgE; cytokines such as IL1; cytokine receptors such as IL2 receptor; blood group antigens; flk2/flt3 receptor; obesity (OB) receptor; mpl receptor; CTLA-4; protein C; neutropilins; ephrins and receptors; netrins and receptors; slit and receptors; chemokines and chemokine receptors such as CCL5, CCR4, CCR5; amyloid beta; complement factors, such as complement factor D; lipoproteins, such as oxidized LDL (oxLDL); lymphotoxins, such as lymphotoxin alpha (LTa). Other molecular targets include Tweak, B7RP-1, proprotein convertase subtilisin/kexin type 9 (PCSK9), sclerostin, c-kit, Tie-2, c-fms, and anti-M1.

Exemplary glycoprotein products may include abatacept (ORENCIA®, Bristol-Myers Squibb), abciximab (REOPRO®, Roche), adalimumab (HUMIRA®, Bristol-Myers Squibb), aflibercept (EYLEA®, Regeneron Pharmaceuticals), alefacept (AMEVIVE®, Astellas Pharma), alemtuzumab (CAMPATH®, Genzyme/Bayer), atezolizumab (TECENTRIQ®, Genentech), basiliximab (SIMULECT®, Novartis), belatacept (NULOJIX®, Bristol-Myers Squibb), belimumab (BENLYSTA®, Glaxo SmithKline), bevacizumab (AVASTIN®, Roche), canakinumab (ILARIS®, Novartis), brentuximab vedotin (ADCETRIS®, Seattle Genetics), certolizumab (CIMZIA®, UCB, Brussels, Belgium), cetuximab (ERBITUX®, Merck-Serono), daclizumab (ZENAPAX®, Hoffmann-La Roche), denileukin diftitox (ONTAK®, Eisai), denosumab (PROLIA®, Amgen; XGEVA®, Amgen), eculizumab (SOLIRIS®, Alexion Pharmaceuticals), efalizumab (RAPTIVA®, Genentech), etanercept (ENBREL®, Amgen-Pfizer), faricimab (VABYSMO®, Roche), gemtuzumab (MYLOTARG®, Pfizer), golimumab (SIMPONI®, Janssen), ibritumomab (ZEVALIN®, Spectrum Pharmaceuticals), infliximab (REMICADE®, Centocor), ipilimumab (YERVOYTM, Bristol-Myers Squibb), muromonab (ORTHOCLONE OKT3®, Janssen-Cilag), natalizumab (TYSABRI®, Biogen Idee, Elan), ocrelizumab (OCREVUS®, Genentech), ofatumumab (ARZERRA®, Glaxo SmithKline), omalizumab (XOLAIR®, Novartis), palivizumab (SYNAGIS®, MedImmune), panitumumab (VECTIBIX®, Amgen), ranibizumab (LUCENTIS®, Genentech), rilonacept (ARCALYST®, Regeneron Pharmaceuticals), rituximab (MABTHERA®, Roche), tocilizumab (ACTEMRA®, Genentech; RoActemra, Hoffman-La Roche), tositumomab (BEXXAR®, Glaxo SmithKline), and trastuzumab (HERCEPTIN®, Roche).

The term “erythropoietic composition” as used herein refers to a composition comprising at least one erythropoiesis-stimulating glycoprotein that contains glycosylation sites, and among which at least some of the molecules carry a sugar chain, optionally comprising at least one terminal sialic residue (i.e. such molecules are “sialylated”). Similarly, the term “glycoprotein composition” as used herein refers to a composition comprising at least one glycoprotein molecule, among which at least some of the molecules are optionally sialylated.

As used herein, “erythropoiesis-stimulating glycoprotein” means a protein that directly or indirectly causes activation of the erythropoietin receptor, for example, by binding to and causing dimerization of the receptor. Erythropoiesis-stimulating glycoproteins include erythropoietin and variants, analogs, or derivatives thereof that bind to and activate erythropoietin receptor; antibodies that bind to erythropoietin receptor and activate the receptor; or peptides that bind to and activate erythropoietin receptor. The variants, analogs, or derivatives of erythropoietin as meant herein comprise at least three N-glycosylation sites. In one embodiment, the N-glycosylation sites are Asn24, Asn38 and Asn83. Erythropoiesis-stimulating glycoproteins include, but are not limited to, epoetin alfa, epoetin beta, epoetin delta, epoetin omega, epoetin iota, epoetin zeta, and analogs thereof, pegylated erythropoietin, carbamylated erythropoietin, mimetic peptides (including EMPI/hematide), and mimetic antibodies. Exemplary erythropoiesis stimulating glycoproteins include erythropoietin, darbepoetin, erythropoietin agonist variants, and peptides or antibodies that bind and activate erythropoietin receptor (and include compounds reported in U. S. Patent Application Publication Nos. 2003/0215444 20 and 2006/0040858, the disclosures of each of which is incorporated herein by reference in its entirety) as well as erythropoietin molecules or variants or analogs thereof as disclosed in the following patents or patent applications, which are each herein incorporated by reference in its entirety: U.S. Pat. Nos. 4,703,008; 5,441,868; 5,547,933; 5,618,698; 5,621,080; 5,756,349; 5,767,078; 5,773,569; 25 5,955,422; 5,830,851; 5,856,298; 5,986,047; 6,030,086; 6,310,078; 6,391,633; 6,583,272; 6,586,398; 6,900,292; 6,750,369; 7,030,226; 7,084,245; 7,217,689; PCT publication nos. WO 91/05867; WO 95/05465; WO 99/66054; WO 00/24893; WO 01/81405; WO 00/61637; WO 01/36489; WO 02/014356; WO 02/19963; WO 02/20034; WO 02/49673; WO 02/085940; WO 03/029291; WO 2003/055526; WO 2003/0844 77; WO 2003/094858; WO 2004/002417; WO 2004/002424; WO 2004/009627; WO 2004/024761; WO 2004/033651; WO 2004/035603; WO 2004/043382; WO 2004/101600; WO 2004/101606; WO 2004/101611; WO 2004/1063 73; WO 2004/018667; WO 2005/001025; WO 2005/001136; WO 2005/021579; WO 2005/025606; WO 2005/032460; WO 2005/051327; WO 2005/063808; WO 2005/063809; WO 2005/070451; WO 2005/081687; WO 2005/084711; WO 2005/103076; WO 2005/100403; WO2005/092369; WO 2006/50959; WO 2006/02646; WO 2006/29094; and US publication nos. US2002/0155998; US2003/0077753; US2003/0082749; US 2003/0143202; US2004/0009902; US2004/0071694; US2004/0091961; US2004/0143857; US2004/0157293; US2004/0175379; US2004/0175824; US2004/0229318; US2004/0248815; US2004/0266690; US2005/0019914; US2005/0026834; US2005/0096461; US2005/0107297; US2005/0107591; US2005/0124045; US2005/0124564; US2005/0137329; US2005/0142642; US2005/0143292; US2005/0153879; US2005/0158822; US2005/0158832; US2005/0170457; US2005/0181359; US2005/0181482; US2005/0192211; US2005/0202538; US2005/0227289; US2005/0244409; US2006/0088906; US2006/0111279.

“Erythropoietin”, “erythropoietin polypeptide” or “EPO” refers to a glycoprotein that belongs to the group of “erythropoiesis-stimulating glycoproteins” and directly or indirectly causes activation of the erythropoietin receptor, for example, by binding to and causing dimerization of the receptor. It refers to a glycoprotein, having the amino acid sequence set out in SEQ ID NO: 1. In one embodiment, this term includes an amino acid sequence substantially homologous to the sequence of SEQ ID NO: 1, whose biological properties relate to the stimulation of red blood cell production and the stimulation of the division and differentiation of committed erythroid progenitors in the bone marrow. As used herein, these terms include such proteins modified deliberately, as for example, by site directed mutagenesis or accidentally through mutations. “Erythropoietin” as used herein includes erythropoietin and variants, analogs, or derivatives thereof that bind to and activate erythropoietin receptor. The variants, analogs, or derivatives of erythropoietin as meant herein comprise at least three N-glycosylation sites, in one embodiment the N-glycosylation sites are Asn24, Asn38 and Asn83. Erythropoietins include, but are not limited to, epoetin alfa, epoetin beta, epoetin delta, epoetin omega, epoetin iota, epoetin zeta, and analogs thereof, pegylated erythropoietin, and carbamylated erythropoietin. In an embodiment, the terms erythropoietin or EPO analog include analogs having from 1 to 6 additional sites for glycosylation, analogs having at least one additional amino acid at the carboxy terminal end of the glycoprotein, wherein the additional amino acid includes at least one glycosylation site, and analogs having an amino acid sequence which includes a rearrangement of at least one site for glycosylation. As used herein, “rearrangement” of a glycosylation site means the deletion of one or more glycosylation sites in naturally occurring EPO and the addition of one or more non-naturally occurring glycosylation sites. These terms include both natural and recombinantly produced human erythropoietin.

The term “batch”, “cultivation batch” or “fermentation batch” as used herein refers to a glycoprotein composition that was obtained by performing a method for recombinantly producing a glycoprotein composition in one bioreactor once. In particular, it refers to a glycoprotein composition that was obtained by performing once a method comprising the steps of
(a) providing a bioreactor comprising a pH measuring device positioned in the bioreactor and configured to be in physical contact with a cultivation medium,
(b) closing and sterilizing the bioreactor,
(c) filling the cultivation medium into the bioreactor,
(d) calibrating the pH measuring device,
(e) inoculating the bioreactor with a recombinant host cell which expresses the glycoprotein,
(f) cultivating the recombinant host cell under conditions suitable for producing the glycoprotein, and
(g) thereby producing the glycoprotein.

The obtained glycoprotein composition may subsequently be split into smaller volumes for further processing, which will commonly be referred to as “of/from the same batch”, and which, unless subjected to different subsequent processing steps, will be of comparable quality with regard to physical and chemical parameters, such as e.g. glycosylation profile.

The terms “glycosylation” and “glycosylated” as used herein refers to the presence of a carbohydrate (e.g., an oligosaccharide or a polysaccharide, also referred to as a “glycan”) attached to biological molecule (e.g., a protein or a lipid). In particular embodiments, glycosylation refers to the presence of a glycan (e.g., an N-Glycan) attached to a protein (e.g., an erythropoiesis-stimulating glycoprotein, in particular erythropoietin) or a portion of a protein of interest. As used herein, the term “glycan” refers to a polysaccharide, oligosaccharide or monosaccharide. Glycans can be monomers or polymers of sugar residues and can be linear or branched. N-linked glycosylation refers to the attachment of the carbohydrate moiety to the side-chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine can also be involved in O-linked glycosylation. For a review of glycosylation, see, e.g., Varki et al., Essentials of Glycobiology, 3^{rd} Edition, Cold Spring Harbor Laboratory Press, 2015-2017. The terms “aglycosylated” and “not glycosylated,” as used interchangeably herein, refer to a protein or a portion of a protein of interest that is not glycosylated (e.g., not N-glycosylated). A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′ sulfo N-acetylglucosamine, etc). Glycan is also used herein to refer to the carbohydrate portion of a glycoconjugate, such as a glycoprotein, glycolipid, glycopeptide, glycoproteome, peptidoglycan, lipopolysaccharide or a proteoglycan. Glycans usually consist solely of O-glycosidic linkages between monosaccharides. For example, cellulose is a glycan (or more specifically a glucan) composed of B-1,4-linked D-glucose, and chitin is a glycan composed of β-1,4-linked N-acetyl-D-glucosamine. Glycans can be homo or heteropolymers of monosaccharide residues, and can be linear or branched. Glycans can be found attached to proteins as in glycoproteins and proteoglycans. They are generally found on the exterior surface of cells. O- and N-glycans are very common in eukaryotes but may also be found, although less commonly, in prokaryotes. As used herein, a “galactosylated glycan” refers to a glycan that includes at least one galactose sugar residue. In some embodiments, a galactosylated glycan is a G1, G2, G1F, G2F, A1, and/or A2 glycan. In some embodiments, a galactosylated glycan includes one or more poly N-acetyl lactosamine repeats. In some embodiments, a galactosylated glycan is a galactose-alpha-1-3-galactose-containing glycan. In some embodiments, a galactosylated glycan is a tri-antennary glycan or a tetra-antennary glycan. A non-galactosylated glycan includes G0F or G0.

As used herein, the term “N-Glycan” refers to an N-linked oligosaccharide, e.g., one that is attached by an asparagine N-acetylglucosamine linkage to an asparagine residue of a polypeptide. N-Glycans are found attached to the R-group nitrogen (N) of asparagine in the sequon. The sequon is a Asn-X-Ser or Asn-X-Thr sequence, where X is any amino acid except proline. N-Glycans have a common pentasaccharide core of Man3GlcNAc2 (“Man” refers to mannose; “Glc” refers to glucose; and “NAc” refers to N-acetyl; “GlcNAc” refers to N-acelylglucosamine). The pentasaccharide core may be fucosylated. The term “trimannose core” used with respect to the N-Glycan also refers to the structure Man3GlcNAc2 (“Man3”). N-glycans differ with respect to the number of branches (antennae) comprising peripheral sugars (e.g., fucose [herein abbreviated as “Fuc”] and sialic acid) that are added to the Man3 core structure. N-Glycans are classified according to their branched constituents (e.g., high mannose, complex or hybrid). Abbreviations used herein, including abbreviations of sugars, are of common usage in the art. Other common abbreviations include “PNGase”, which refers to peptide N-glycosidase F (EC 3.2.2.18). The substrate UDP-GlcNAc is the abbreviation for UDP-N-acetylglucosamine. The intermediate ManNAc is the abbreviation for N-acetylmannosamine. The intermediate ManNAc-6-P is the abbreviation for N-acetylmannosamine-6-phosphate. The intermediate Sia-9-P is the abbreviation for sialate-9-phosphate. The intermediate Cytidine monophosphate-sialic acid is abbreviated as “CMP-Sia.” Sialic acid is abbreviated as “Sia,” “Neu5Ac” “NeuAc” or “NANA”.

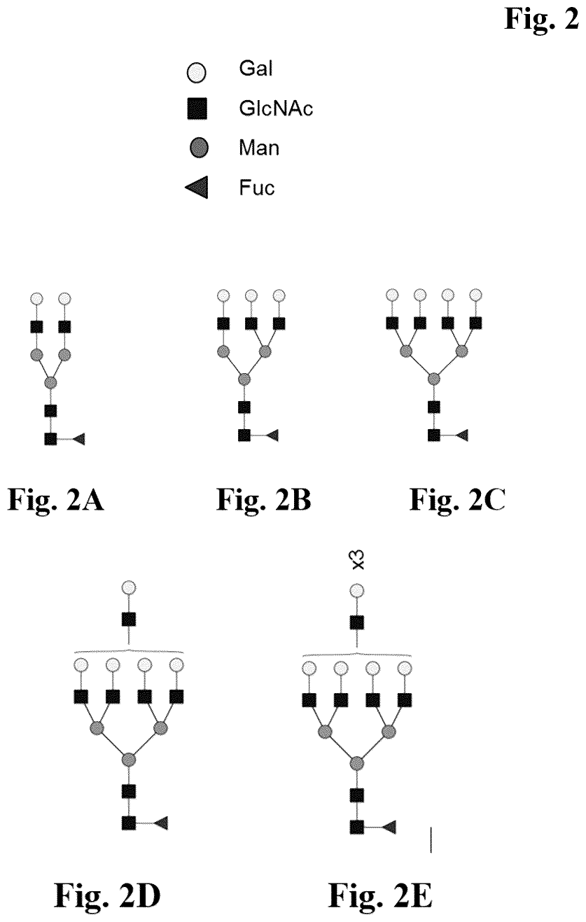

The N-Glycans on erythropoiesis-stimulating glycoproteins, e.g. erythropoietin, include one or more N-acetyl lactosamine units bound to the pentasaccharide core structure of the N-linked oligosaccharide. The number of oligosaccharide “branches” as used herein refers to the number of individual oligosaccharide chains bound to the pentasaccharide core structure. For instance, as demonstrated in FIG. 2A, two individual oligosaccharide chains are bound to the pentasaccharide core structure, hence, the N-Glycan comprises two branches and is biantennary. For instance, as demonstrated in FIG. 2B, three individual oligosaccharide chains are bound to the pentasaccharide core structure, hence, the N-Glycan comprises three branches and is triantennary. For instance, as demonstrated in FIG. 2C, four individual oligosaccharide chains are bound to the pentasaccharide core structure, hence, the N-Glycan comprises four branches and is tetraantennary. The N-Glycans on erythropoiesis-stimulating glycoproteins, e.g. erythropoietin, include poly N-acetyl lactosamine units bound to the pentasaccharide core structure of the N-linked oligosaccharide. The term “repeat” or “poly N-acetyl lactosamine repeat” as used herein refers to the number of N-acetyl lactosamine units within one oligosaccharide branch minus one for the first N-acetyl lactosamine unit. For instance, as demonstrated in FIG. 2D two N-acetyl lactosamine units are present within one oligosaccharide branch, meaning that the oligosaccharide comprises one poly N-acetyl lactosamine repeat. The corresponding N-Glycan structure herein is also referred to as “tetraantennary+1 repeat”. For instance, as demonstrated in FIG. 2E, four N-acetyl lactosamine units are present within one oligosaccharide branch, meaning that the oligosaccharide comprises three poly N-acetyl lactosamine repeats. The corresponding N-Glycan structure herein is also referred to as “tetraantennary+3 repeats”.

The term “glycosylation occupancy” as used herein refers to the probability that a protein is glycosylated at a particular glycosylation site (e.g., an Asn residue of a consensus glycosylation site) or the relative content of proteins in a population of proteins that are glycosylated at a particular glycosylation site. For example, an erythropoietin polypeptide may be glycosylated on amino acid residues Asn24, Asn38 and/or Asn83 of SEQ ID NO:1.

The terms “host cell”, “host cell line”, and “host cell culture” are used interchangeably and refer to cells into which an exogenous nucleic acid has been introduced, including the progeny of such cells. The person skilled in the art is aware of methods for making (erythropoiesis-stimulating) glycoprotein compositions, wherein the method comprises cultivating a host cell comprising nucleic acid(s) encoding the (erythropoiesis-stimulating) glycoprotein, as provided above, under conditions suitable for expression of the (erythropoiesis-stimulating) glycoprotein, and optionally recovering the (erythropoiesis-stimulating) glycoprotein from the host cell (or host cell culture medium), as well as of methods for making host cells which comprise nucleic acid(s) encoding a (erythropoiesis-stimulating) glycoprotein. For recombinant production of an (erythropoiesis-stimulating) glycoprotein composition, nucleic acids encoding the (erythropoiesis-stimulating) glycoprotein, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. Mammalian cell lines that are adapted to grow in suspension may be useful. Examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as YO, NSO and Sp2/0. In one embodiment of the invention, the host cell is a CHO cell. In one embodiment of the invention, the host cell is a CHO-K1 cell. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

The terms “in-line (pH) measurement” refers to a measurement that occurs directly in the bioreactor with a process sensor that is usually installed in the bioreactor. The terms “in-line (pH) probe” or “in-line (pH) measurement device” as used herein refer to such a process sensor that is installed in the bioreactor. The generated measurements may be sent in real time to systems for automated control of the measured parameter, such as pH value. Cell culture process parameters such as pH, dissolved oxygen, dissolved CO₂, temperature and conductivity are commonly measured in-line.

The terms “sialylation” and “sialylated” refers to the presence of sialic acid on a protein or a portion of a protein of interest, particularly as a component of a glycan (e.g., N-Glycan) chain attached to a protein. Sialic acid (also referred to herein as a “sialic acid moiety”) refers generally to N- or O-substituted derivatives of neuraminic acid. N-acetylneuraminic acid (5-Acetamido-3,5-dideoxy-D-glycero-D-galactononulosonic acid; also known as NANA or Neu5Ac) is the most common sialic acid in mammals. Other exemplary sialic acids include, without limitation, 2-keto-3-deoxy-D-glycero-D-galactonononic acid (also known as Kdn), N-glycolylneuraminic acid (also known as Neu5Gc or NGNA), neuraminic acid (also known as Neu), and 2-deoxy-2,3-didehydro-Neu5Ac (also known as Neu2en5Ac). Free sialic acid (Sia) can be used for glycan synthesis after activation onto the nucleotide donor CMP-Sia. Transfer of Sia from CMP-Sia onto newly synthesized glycoconjugates (e.g., glycoproteins) in the Golgi system of eukaryotes is catalyzed by a family of linkage-specific sialyl-transferases (STs). Sialic acids are typically the terminating residues of glycan (e.g., N-Glycan) branches. In some embodiments, sialic acids can occupy internal positions within glycans, most commonly when one sialic acid residue is attached to another. For a review of sialylation and sialic acid, see, e.g., Chapter 15 of Varki et al., Essentials of Glycobiology, 3rd Edition, Cold Spring Flarbor Laboratory Press, 2015-2017.

The term “sialic acid content” as used herein refers to the level or amount of sialylation of a glycosylated protein or a portion of a glycosylated protein of interest. The term “average sialic acid content” with respect to a composition containing a glycoprotein (e.g., a pharmaceutical composition or a batch) refers to the total number of moles of sialic acid in the composition per mole of glycoprotein in the composition. Thus, for example, such a composition may contain a heterogeneous pool of glycoproteins with individual glycoproteins within the composition having varying levels of sialylation (e.g., in the range of 0-14 moles of sialic acid per mole of erythropoiesis-stimulating glycoprotein).

TABLE 1
Examples of common isoforms of erythropoietin

| Erythropoietin Isoform | Number of sialic acid residues |
|---|---|
| Isoform 2 | 14 |
| Isoform 3 | 13 |
| Isoform 4 | 12 |
| Isoform 5 | 11 |
| Isoform 6 | 10 |
| Isoform 7 | 9 |
| Isoform 8 | 8 |

With regard to sialylation, erythropoietin exists mainly in ten isoforms. The term “Isoform” refers to a group of erythropoietin molecules which have an identical amino acid sequence and the same number of bound sialic acid residues. The isoforms have the same isoelectric point and may differ with regard to the extent, complexity and antennarity of the glycosyl residues bound to the amino acid sequence. For example, the term “Isoform 2 of erythropoietin” encompasses a group of erythropoietin molecules which have 14 sialic acid residues. Isoform 3 has 13 sialic acids and so on. In addition, there are rare forms of erythropoietin which have additional sialic acids which are not at the terminus. Thus, Isoform 1 has 15 and Isoform l′ has 16 sialic acids bound to the glycosyl residues.

The term “sialic acid free” as used herein refers to a population of glycoproteins that is substantially free from N-Glycans comprising terminal sialic acid moieties. In one embodiment, the term “sialic acid free” as used herein refers to a population of glycoproteins comprising a relative frequency of N-linked glycans that include a sialic acid moiety of 5% and less. In one embodiment, the sialic acid free glycoprotein comprises a relative frequency of N-linked glycans that include a sialic acid moiety of about 0%.

The term “glycosylated variant” or “glycosylation variant” as used herein refers to a glycoprotein, in particular an erythropoiesis-stimulating glycoprotein, e.g. erythropoietin, that is characterized by a defined carbohydrate moiety attached thereto. A “glycosylated variant” or “glycosylation variant” of a glycoprotein may be a glycoprotein which comprises a specific N-linked oligosaccharide, in particular an N-Glycan with a pentasaccharide core of Man3GlcNAc2 and with different numbers of branches (antennae) bound to the pentasaccharide core structure. In certain embodiments of all aspects and embodiments, the “glycosylated variants” may be triantennary or tetraantennary. In another aspect, the “glycosylated variant” may include differing numbers of poly N-acetyl lactosamine units bound to the pentasaccharide core structure of the N-linked oligosaccharide (“repeat”/“poly N-acetyl lactosamine repeat”). In particular, the “glycosylated variants” of a glycoprotein may have a “triantennary+1 repeat”, a “tetraantennary+1 repeat”, a “tetraantennary+2 repeats” and/or a “tetraantennary+3 repeats” N-glycan structure. The term “glycosylated variant” or “glycosylation variant” herein also comprises “sialylated variants” of a glycoprotein. A “sialylated variant” or “sialylation variant” of a glycoprotein as used herein refers an glycoprotein with a specific level or amount of sialylation (e.g., in the case of an erythropoiesis stimulating glycoprotein, this may be the range of 0-14 moles of sialic acid per mole of erythropoiesis-stimulating glycoprotein). A “glycosylated variant” or “glycosylation variant” of a glycoprotein may thus also be a glycoprotein which comprises a specific amount of sialic acid moieties. In a particular embodiment, a glycosylated variant of a erythropoiesis glycoprotein may be of one of the main ten isoforms of erythropoietin (Isoform 1-10), particularly one of the seven isoforms shown in Table 1. A glycoprotein composition may typically be comprised of a mixture of different glycosylated variants of a glycoprotein having defined relative amounts for the different glycosylated variants present in the composition. In the case of an erythropoietic composition, this may be a mixture of different glycosylated variants of an erythropoiesis-stimulating glycoprotein having defined relative amounts for the different glycosylated variants present in the composition.

The term “fed-batch” as used herein relates to a cell culture in which the cells are fed continuously or periodically with a feed medium containing nutrients. The feeding may start shortly after starting the cell culture on day 0 or more typically one, two or three days after starting the culture. Feeding may follow a preset schedule, such as every day, every two days, every three days etc. Alternatively, the culture may be monitored for cell growth, nutrients or toxic by-products and feeding may be adjusted accordingly. Typically, a batch or fed-batch culture is stopped at some point and the cells and/or the protein of interest in the medium are harvested and optionally purified.

The term “media specific correlation” refers to a mathematical description of a relationship between the pH value in a given cultivation medium within a bioreactor and the corresponding CO₂ concentration determined in the headspace and/or the exhaust of the bioreactor. The media specific correlation is universal for the respective medium and applicable, independent of location and the vessel used. The media specific correlation for a specific cultivation medium can be determined by placing said cultivation medium in a vessel and determining the exhaust CO₂ concentration at different pH values and use this data set for developing a suitable model. As the correlation is scale-independent, practically any vessel, such as a simple tank or a bioreactor, can be used for collecting such a data set. Preferably, several measurements are taken to provide a more robust dataset. The skilled person is familiar with methods to deduce correlations from such data sets. According to embodiments, the Media specific correlation is an equation FCO2_{M1}(pH)-REL-M1 (pH) obtained by mathematically fitting multiple empirically determined pairs of a pH-value of a sample of the cultivation medium used in the bioreactor and a respectively measured fraction of CO₂ gas in the headspace above said sample (which may be measured directly in the headspace or, more commonly, in the exhaust of the bioreactor). The skilled person is familiar with methods for mathematically fitting empirically determined data sets, e.g. linear, quadratic or logarithmic fitting. In one embodiment, the data set is quadratically fitted. The sample may be, for example, the totality of a cell free cultivation medium in a bioreactor or a cell free aliquot of said cultivation medium.

The quality of the model, that is, the ability of the model to describe the data points modeled may, as one example, be described by the regression coefficient R-squared (R²). An R² of 1 means that all data points are perfectly described by the model. In all models prepared for the present method, R-squared was found to be larger than 0.98. The root mean squared error was 0.02 in all models. Taking into account potential errors of calibration, environmental factors and the like, the accuracy of the present method is considered to be within 0.03 pH units of the pH of the cultivation medium.

The term “offset” as used herein refers to the sum of the errors, as compared to the actual pH value in the cultivation medium when determining a pH value from an off-line sample caused by multiple factors, such as sampling procedures, equipment, sample hold times, shifts in sample temperature, carbon dioxide degassing of the sample, inter-device differences, general sample properties (such as cell density, dissolved carbon dioxide and media buffering), and/or cross site/scale pressure effects.

The term “pH measuring device” or “pH probe” refers to a device and/or substance used for measuring a current pH value in a medium. In one embodiment, a pH measuring device is, for example, a potentiometric apparatus. According to preferred embodiments, the pH measuring device is a pH-meter. The pH-meter can be, for example, a continuous pH-meter, i.e., a pH-meter capable of continuously and repeatedly measuring the pH of the cultivation medium in a bioreactor without having to draw samples and without having to insert said pH-meter in the medium for each individual measurement. For example, a pH measuring device can be a precise voltmeter, in contact with the cultivation medium and connected to a reference electrode, and scaled in such a way that it displays not the measured potential, but the ready pH value. Preferentially, the pH measuring device is immersed in the medium and is used for repeatedly or continuously measuring the current pH value in the cultivation medium during the whole time while cultivating cells in the bioreactor. For example, the pH measuring device may measure a current pH value every minute, or every 30 minutes, or every hour. In typical today's pH-meters used as pH measuring devices, a reference electrode is built into the pH electrode, which makes the device compact.

The term “calibration” or “calibrating” as used herein refers to the procedure of comparing a measurement value, such as a pH value, of a measurement device, such as a pH measurement device, to a reference measurement value or to a calibration standard of known accuracy and nominal (e.g. a pH) value, and adjusting the measurement device to that reference value. A pH measurement device is typically calibrated using so-called calibration buffers of defined pH value. Commonly, one, two or three buffers with different pH values may be used (one-point, two-point, and three-point calibration, respectively). This procedure requires the contacting of the pH measuring device with the calibration buffer(s). As this method of calibrating would break the sterility of a sterilized bioreactor, a common alternative way for calibrating inline pH measurement devices installed in bioreactors would be to take a sample from the bioreactor and measuring the sample's pH offline with another pH measurement device of known accuracy, and then one-point calibrating the inline pH measurement device to the pH value that was measured offline (“off-line measurement”).

The term “carbon dioxide-based pH reference method”, “carbon dioxide-based calibration” or “carbon dioxide-based calibrating” refers to the method disclosed herein that obviates the need for sampling and measuring pH offline by calculating the pH value in the cultivation medium utilizing a media specific correlation between carbon dioxide concentration in the gas phase and dissolved carbon dioxide, bicarbonate and dependent proton concentrations that directly affects the pH value in carbonate buffered systems. Said carbon dioxide-based pH reference method is independent of scale and bioreactor configuration and is able to determine accurately the pH in a cultivation medium in a bioreactor without the need to take samples.

The term “pH value of/in the cultivation medium” relates to the pH value that has been determined by an in-line pH measurement device that is in direct contact with the cultivation medium and therefore has no offsets introduced by e.g. sampling procedures, equipment, sample hold times, shifts in sample temperature and carbon dioxide degassing, inter-device differences, sample properties (cell densities, dissolved carbon dioxide concentrations, media buffering and the like), and/or cross site/scale pressure effects, and that has been calibrated according to accepted standards, e.g. with certified pH calibration standards 4.01, 7.00, 9.21 pH. In an embodiment of all aspects, to determine the pH value of/in the cultivation medium with maximum accuracy, two independent pH measurement devices that have been calibrated to said standards with a maximum acceptable error of 0.01 pH, may be put in direct contact with the cultivation medium simultaneously. The maximum acceptable difference of the values measured in the cultivation medium with the above-mentioned two independent pH measurement devices may be set to 0.02 pH units, and averages of the two measured values may be used.

As used herein, the term “relative content” of a glycosylated variant refers to the amount of a particular glycosylated variant in a glycoprotein composition. Typically, glycoprotein compositions may be heterogeneous and may contain two or more glycosylated variants, and, therefore, no single glycosylated variant makes up 100% of the composition. Relative content can be expressed in any of a variety of forms known in the art; for example, relative content can be expressed as a relative content of the total amount of glycoprotein in a composition, or can be expressed relative to the amount of a particular type of glycosylation, e.g. relative to the total amount of N-glycan variants or to the total amount of sialylated isoforms. Methods and assays for determining the relative content of glycosylated variants are described herein.

The term “sterilization” as used herein refer to any process that eliminates (removes) or kills (deactivates) all forms of life and other biological agents. Sterilization can be achieved with one or more of the following: heat, chemicals, irradiation, high pressure, and filtration. When a system or system element cannot be moved (e.g. a large stainless steel bioreactor), an in situ sterilization process called Sterilization-in-place (SIP) is used. SIP processes may reduce or eliminate post-sterilization handling by providing aseptic connections between sterilized equipment. Common modes of sterilization for SIP are steam (moist heat), superheated water, dry heat, gas, liquid, and vapor sterilization, with steam being the most common method. SIP methods are generally used with closed systems, such as large-scale biotech manufacturing in a GMP environment, to maintain aseptic conditions during product manufacturing. The term “under sterile conditions” as used herein refers to conditions that avoid and/or prevent the contamination of the bioreactor (including all vessels and devices that attached to it), the cultivation medium and/or the cell culture with any undesired live organisms, such as, but not limited to, any germs, pathogens, microorganisms (such as bacteria) including their spores, and/or viruses (but of course not including the recombinant host cell which expresses a glycoprotein of interest), after sterilization of the bioreactor has taken place. Methods for recombinantly producing a glycoprotein “under sterile conditions” are well known to the person skilled in the art and include the use of closed systems as described above, and suitable measures to ensure that any materials, whether they be solid, liquid or gaseous, or any devices that come into contact with the cultivation medium and/or the inside of the bioreactor during the manufacturing process, in particular during the cultivation process, (such as gas mixtures used for aeration, feed streams, sampling devices) are free from any contaminating live organisms (except the host cell).

The term “batch-to-batch variability” as used herein, refers to the difference in properties between an isolated glycoprotein composition obtained by one run of a recombinant production method and another isolated glycoprotein composition obtained by a subsequent run of the same recombinant production method. Said batch-to-batch variability may be quantified with regard to any chemical or physical property of the glycoprotein composition, such as glycoprotein concentration, pH value, osmolality, glycosylated variant content, or protein modifications (e.g. deamidation, acidic variants). In one aspect of the invention, batch-to-batch variability is determined with regard to the relative content of at least glycosylated variant, e.g. N-glycan variants or sialylated isoforms.

The term “yield” as used herein refers to the amount of glycoprotein which is harvested from the recombinant host cell culture. Yield may be presented by “mg protein/g biomass” (measured as dry cell weight or wet cell weight) of a host cell. The absolute yield of a fermentation batch may be represented by “g protein” and may be calculated by determining the concentration of the recombinant glycoprotein composition using a suitable method, such as RP-HPLC, and by multiplying the resulting concentration with the volume of the obtained glycoprotein composition. The term “titer” when used herein refers similarly to the amount of produced protein of interest or model protein, presented as “g protein/L cultivation medium”.

### 2. Methods of the Invention

In commercial production of recombinant (glyco-)proteins, pH probes are commonly placed inside the bioreactor to measure pH inline. At the beginning of the cell culture process, the bioreactor is closed and sterilized, before filling it with a cultivation medium. The sterilization of the bioreactor however has an impact on the in-line pH probe that is positioned in the bioreactor, as the temperatures used for SIP are usually above pH probe working range and in a range where the electrolyte within the probe may liquefy, leading to potential asymmetry changes. In order to be able to rely on the measurements shown by the in-line pH probe, a re-calibration is thus necessary after sterilization. However, in GMP (good manufacturing practices) manufacturing, after sterilizing and filling, it is no longer possible to directly equilibrate or re-adjust the inline pH probes without breaking sterility and violating GMP principles. The pH value of the cell culture is thus commonly monitored and controlled using sample-based offline measurements. For this, samples from the medium are taken after sterilization of the bioreactor to measure pH offline (e.g. with a glass electrode or a blood gas analyzer), and in-line pH probes are then re-adjusted (calibrated) based on the offline pH value. During the cultivation, offline sampling is frequently continued in order to detect any drifts in the inline pH probe. For that, the offline measured pH value is continually compared to the inline measured pH value, and if they show a difference beyond a certain acceptance limit, the inline pH probe is re-calibrated to the value that was measured in the offline sample.

This procedure, however, can lead to problems. Various factors, such as measurement inaccuracies, wrong handling of the buffer solutions (e.g. not using fresh alkaline buffer solutions as CO₂ that has absorbed from the ambient air leads to an decrease in pH), extreme pH values >pH 12 or <pH 2, sample hold time leading to shifts in pH, sample temperature or degassing of carbon dioxide, use of different offline methods, or different sampling procedures or equipment, can influence the pH that is measured off-line which thus deviates from the actual “true” pH within the bioreactor. All these effects can add up to an offset of up to +/−0.15 pH units. As a consequence, recalibrating in-line pH electrodes based on sample-based off-line measurements introduces significant offsets compared to the actual pH inside the bioreactor, i.e., the measured pH value does not accurately reflect the pH in the cultivation medium. Errors may not only be introduced at the re-adjustment of pH electrodes after sterilization and before the inoculation of the cultivation medium with the host cells, leading to an inconsistency of processes with varying pH. If offline measurement is used to continue monitoring and re-adjusting the inline pH probes during the cell culture process, the cells' metabolism inside the bioreactor adds to the pH offset as well by changing the composition of the medium.

This can also affect the control of the cell culture process. Commonly, a fixed pH set point is defined for a cell culture process for producing a therapeutic protein, and in order to keep the pH in the cell culture constant, most commonly, the cultivation medium is controlled either via the partial pressure of dissolved carbon dioxide (pCO₂) in the cell culture or by adding base into the cultivation medium. If the measured pH does not reflect the pH in the cultivation medium, the pH may erroneously be set to a pH value that is above or below the desired set point, leading to undesired results.

The inventors have found that by using a method of calibrating an inline pH measurement device in a closed bioreactor to a pH value that causes the inline pH measurement device to reliably reflect the pH value in the cultivation medium and thus avoids the need for offline pH determination resulted in a greatly reduced variability of certain glycosylated variants, and also of the overall yield, when recombinantly producing a glycoprotein in a bioreactor using mammalian host cell culture. It has surprisingly been found that a significant improvement with regard to the variability of glycosylated variant of a recombinantly produced glycoprotein composition could be achieved, without changing other parameters, solely by addressing the variability that using an offline, sample-based pH measurement/calibration method confers to a (large scale) cultivation process by replacing it with a highly accurate carbon dioxide-based method for determining the pH value in a cultivation medium and adjusting an inline pH probe to that pH value.

This can be achieved for instance by using a non-invasive method to determine pH and pCO₂ in a sterile bioreactor that obviates the need to sample and measure offline. This method utilizes the chemical correlation between carbon dioxide in the gas phase and dissolved carbon dioxide, bicarbonate and dependent proton concentrations that directly affect the pH in carbonate buffered systems. Said carbon dioxide-based pH reference method is independent of scale and bioreactor configuration and is able to determine accurately the true pH in the bioreactor without the need to take samples.

In case of commercial manufacture of therapeutic glycoproteins, the reduced variability not only results in a highly consistent product quality with regard to glycosylated variants, but also improves overall process yield and efficiency as it reduces the risk of not meeting the required product specification, thus avoiding the need to discard partial or entire production batches that were not within the specification.

One aspect of the invention is thus a method for recombinantly producing a glycoprotein composition comprising at least one glycoprotein, wherein the method comprises the step of
cultivating a recombinant host cell which expresses the glycoprotein in a cultivation medium in a bioreactor having a pH measuring device positioned in the bioreactor and configured to be in physical contact with the cultivation medium,
characterized in that
(a) the cultivating is performed under sterile conditions,
(b) the pH value measured with the pH measuring device differs by 0.05 units or less from the pH value of the cultivation medium, and
(c) the relative content of at least one glycosylated variant in the glycoprotein composition has reduced batch-to-batch variability, compared to a method wherein the pH value measured by the pH measuring device differs by more than 0.05 units, preferably by more than 0.03 units, from the pH value in the cultivation medium,
and thereby producing the glycoprotein composition.

Another aspect of the invention is a method for recombinantly producing a glycoprotein composition comprising at least one glycoprotein in a recombinant host cell which expresses the glycoprotein, comprising the steps of
(a) providing a bioreactor comprising a pH measuring device positioned in the bioreactor and configured to be in physical contact with a cultivation medium,
(b) closing and sterilizing the bioreactor,
(c) filling the cultivation medium into the bioreactor,
(d) calibrating the pH measuring device,
(e) inoculating the bioreactor with the recombinant host cell,
(f) cultivating the recombinant host cell under conditions suitable for producing the glycoprotein composition, and
(g) thereby producing the glycoprotein composition
characterized in that the calibrating of step (d) comprises the steps of
(i) introducing a gas mixture comprising carbon dioxide gas into the bioreactor,
(ii) determining the carbon dioxide concentration in the headspace and/or the exhaust,
(iii) calculating the pH value of the cultivation medium based on a media specific correlation, and
(iv) adjusting said pH measuring device to the pH value calculated in step (iii).

In certain embodiments of all aspects and embodiments, the pH value measured with the pH measuring device after the calibration step differs by 0.05 pH units or less from the pH value of the cultivation medium. In a preferred embodiment of all aspects and embodiments, the pH measured with the pH measuring device after the calibration step differs by 0.03 pH units or less, from the pH value of the cultivation medium.

In certain embodiments of all aspects and embodiments, the carbon dioxide concentration is determined in the exhaust of the bioreactor.

The improvement in variability may be assessed by calculating the standard deviation of at least one glycosylated variant determined for at least two fermentation batches of the glycoprotein composition (i.e. from at least two different cultivations performed according to the method of the invention), and comparing it to the standard deviation calculated for the same glycosylated variant(s) determined for at least two batches of the glycoprotein composition manufactured according to another method, i.e., a method wherein the pH value measured with the pH measuring device differs by more than 0.05 pH units, preferably by more than 0.03 pH units, from the pH value of the cultivation method. In one embodiment, the batch-to-batch variability is compared by comparing standard variation between batches that have been manufactured with a manufacturing method that is essentially the same except for the method used for calibrating the pH measurement device. In certain embodiments of all aspects and embodiments, the standard deviation is calculated for at least one glycosylated variant from glycoprotein compositions from at least three fermentation batches. In another embodiment, the standard deviation is calculated for at least one glycosylated variant from glycoprotein compositions from at least five fermentation batches. In yet another embodiment, the standard deviation is calculated for at least one glycosylated variant from glycoprotein compositions from at least 10 fermentation batches.

In certain embodiments of all aspects and embodiments, the glycoprotein composition comprises at least one glycosylated variant of the glycoprotein, and the standard deviation of the relative content of the glycosylated variant calculated for glycoprotein compositions from at least two fermentation batches is decreased, compared to the standard deviation of the relative content of the glycosylated variant calculated for glycoprotein compositions which have been produced using a method wherein the pH value measured by the pH measuring device differs by more than 0.05 units, preferably by more than 0.03 units, from the pH value in the cultivation medium.

In one embodiment of all aspects and embodiments, the yield of the glycoprotein composition after the cultivating has reduced batch-to-batch variability, compared to the standard deviation of the yield calculated for glycoprotein compositions which have been produced using a method where the pH value measured by the pH measuring device differs by more than 0.05 units, preferably by more than 0.03 units, from the pH value in the cultivation medium. In one embodiment of all aspects and embodiments, the absolute yield of the glycoprotein composition after the cultivating has reduced batch-to-batch variability, compared to the standard deviation of the absolute yield calculated for glycoprotein compositions which have been produced using a method where the pH value measured by the pH measuring device differs by more than 0.05 units, preferably by more than 0.03 units, from the pH value in the cultivation medium.

In certain embodiments of all aspects and embodiments, the standard deviation of the (absolute) yield calculated for glycoprotein compositions from at least two fermentation batches is decreased, compared to the standard deviation of the (absolute) yield calculated for glycoprotein compositions which have been produced using a method where the pH value measured by the pH measuring device differs by more than 0.05 units, preferably by more than 0.03 units, from the pH value in the cultivation medium. In certain embodiments of all aspects and embodiments, the standard deviation is calculated for glycoprotein compositions from at least three fermentation batches. In another embodiment, the standard deviation is calculated for glycoprotein compositions from at least five fermentation batches. In yet another embodiment, the standard deviation is calculated for glycoprotein compositions from at least ten fermentation batches.

In certain embodiments of all aspects and embodiments, the recombinant host cell is a mammalian cell. In a preferred embodiment of all aspects and embodiment, the recombinant host cell is a CHO cell.

In certain embodiments, the method for recombinantly producing a glycoprotein composition is for commercial purposes and/or is performed using a large-scale bioreactor. In one particular embodiment, the large-scale bioreactor has at least 10 L, preferably at least 50 L, more preferably at least 500 L, particularly preferably at least 5000 L. In certain embodiments of all aspects and embodiments, the bioreactor is for recombinant protein production for commercial purposes. In one embodiment, the large-scale cell culture methods of the invention are suitable for CHO cell culture.

In certain embodiments of all aspects and embodiments, the glycosylated variant is selected from the group consisting of N-glycan variants, O-glycan variants, sialylation variants, mannosylation variants, galactosylation variants and fucosylation variants.

In certain embodiments of all aspects and embodiments, the cultivation medium comprises a carbonate buffer.

In certain embodiments of all aspects and embodiments, the method for recombinantly producing a glycoprotein composition comprises the step of maintaining the pH in the cultivation medium at a desired set point, in particular during the cultivating of the host cells. In certain embodiments of all aspects and embodiments, the desired pH set point in the cultivation medium is adjusted by (a) increasing carbon dioxide influx if pH is above the desired set point, or (b) adding a basic component, preferably 1 mol/L NaOH, to the cultivation medium, if the pH is below the desired set point.

In certain embodiments of all aspects and embodiments, the carbon dioxide is introduced into the bioreactor by sparging.

One further aspect of the invention is a method of reducing the variability of the relative content of at least one glycosylated variant between batches of a recombinant glycoprotein composition, the method comprising:
(a) providing a bioreactor comprising a pH measuring device positioned in the bioreactor and configured to be in physical contact with a cultivation medium,
(b) closing and sterilizing the bioreactor,
(c) filling the cultivation medium into the bioreactor,
(d) calibrating the pH measuring device,
(e) inoculating the bioreactor with a recombinant host cell which expresses the glycoprotein, and
(f) cultivating the recombinant host cell under conditions suitable for producing the glycoprotein, and
(g) thereby producing the glycoprotein,
wherein the calibrating of step (d) comprises the steps of
(i) introducing a gas mixture comprising carbon dioxide gas into the bioreactor
(ii) determining the carbon dioxide concentration in the headspace and/or the exhaust of the bioreactor,
(iii) calculating the pH value of the cultivation medium based on a media specific correlation, and
(iv) adjusting said pH measuring device to the pH value calculated in step (iii), and
wherein steps (a) to (g) result in a first batch of said recombinant glycoprotein with a defined relative content of the glycosylated variant;
(h) repeating the steps (a) to (g) resulting in at least one subsequent batch of said glycoprotein, wherein the relative content of the at least one glycosylated variant of said first and at least one subsequent batch has reduced batch-to-batch variability.

In order to determine the batch-to-batch variability of a parameter, at least two glycoprotein compositions from different fermentation batches have to be assessed. Thus, in certain embodiments of all aspects and embodiments, the reduction of the batch-to-batch variability due to the carbon dioxide-based calibration method is assessed by comparing the variability of glycoprotein compositions from at least two fermentation batches to the variability from at least two fermentation batches that have been obtained with a different method for calibrating the pH measuring device, preferably based on a sample-based offline pH measurement. In certain embodiments of all aspects and embodiments, the reduction of the batch-to-batch variability is assessed by comparing the variability of glycoprotein compositions from at least three fermentation batches. In another embodiment, the reduction of the batch-to-batch variability is assessed by comparing the variability of glycoprotein compositions from at least five fermentation batches. In yet another embodiment, the reduction of the batch-to-batch variability is assessed by comparing the variability of glycoprotein compositions from at least ten fermentation batches.

In a preferred embodiment of all aspects and embodiments, the standard deviation of the relative content of the glycoprotein calculated for said first and at least one subsequent batch is 1% or less, particularly 0.8% or less, most particularly 0.5% or less, of the median of the pH value.

Another aspect of the invention is the use of a carbon dioxide-based method of calibrating a pH measurement device positioned in a bioreactor and configured to be in physical contact with a cultivation medium in the bioreactor for reducing the standard deviation of the relative content of glycosylated variants in a glycoprotein composition calculated between at least two fermentation batches, compared to a glycoprotein composition that has been produced using a pH measurement method where the measured pH value differs by more than 0.05 units, preferably by more than 0.03 units, from the pH value in the cultivation medium. In a preferred embodiment of all aspects and embodiments, the carbon dioxide-based method comprises the steps of (i) introducing a gas mixture comprising carbon dioxide gas into the bioreactor comprising the cultivation medium, (ii) determining the carbon dioxide concentration in the headspace and/or the exhaust of the bioreactor, (iii) calculating the pH of the cultivation medium based on a Media specific correlation, and (iv) adjusting the pH measuring device to the pH value calculated in step (iii). In one embodiment, the use is for reducing the standard deviation compared to a glycoprotein composition that has been produced comprising calibration of the pH measurement device based on a sample-based offline pH measurement.

In certain embodiments of all aspects and embodiments, the cultivation medium is carbonate-buffered. In certain embodiments of the aspect, the carbon dioxide gas is introduced into the bioreactor by sparging.

In certain embodiments of all aspects and embodiments, the glycoprotein composition is an erythropoietic composition. In a preferred embodiment of the aspect, the glycoprotein is an erythropoiesis-stimulating glycoprotein. In certain embodiments of all aspects and embodiments, the glycoprotein composition is erythropoietin.

One aspect of the invention is an erythropoietic composition which has been produced by a method disclosed herein. In certain embodiments of all aspects and embodiments, the erythropoietic composition is characterized in that
(a) about 3.3 area-% to about 3.8 area-% of the N-Glycans of the erythropoiesis-stimulating glycoprotein have biantennary structure;
(b) about 8.6 area-% to about 9.5 area-% of the N-Glycans of the erythropoiesis-stimulating glycoprotein have triantennary structure;
(c about 5.6 area-% to about 5.9 area-% of the N-Glycans of the erythropoiesis-stimulating glycoprotein have triantennary+1 repeat structure;
(d) about 42.2 area-% to about 43.4 area-% of the N-Glycans of the erythropoiesis-stimulating glycoprotein have tetraantennary structure;
(e) about 27.4 area-% to about 28.1 area-% of the N-Glycans of the erythropoiesis-stimulating glycoprotein have a tetraantennary+1 repeat structure;
(f) about 10.7 area-% to about 11.6 area-% of the N-Glycans of the erythropoiesis-stimulating glycoprotein have a tetraantennary+2 repeat structure;
(g) about 13.2 area-% to about 16.0 area-% of the erythropoiesis-stimulating glycoprotein are of Isoform 2;
(h) about 24.1 area-% to about 26.5 area-% of the erythropoiesis-stimulating glycoprotein are of Isoform 3;
   (i) about 23.5 area-% to about 24.6 area-% of the N-Glycans of the erythropoiesis-stimulating glycoprotein are of Isoform 4;
(j) about 17.1 area-% to about 18.6 area-% erythropoiesis-stimulating glycoprotein are of Isoform 5;
(k) about 9.4 area-% to about 11.8 area-% erythropoiesis-stimulating glycoprotein are of Isoform 6;
(l) about 3.7 area-% to about 5.5 area-% of the erythropoiesis-stimulating glycoprotein are of Isoform 7; and/or
(m) about 0.9 area-% to about 1.6 area-% of the erythropoiesis-stimulating glycoprotein are of Isoform 8. In certain embodiments of all aspects and embodiments, the area-percent of N-Glycans of the erythropoiesis-stimulating glycoprotein are determined by anion exchange chromatography after enzymatic release of N-glycosidically-linked oligosaccharides from the glycoprotein and desialylation. In certain embodiments, the anion exchange chromatography is high performance anion-exchange chromatography with pulsed amperometric detection (HPAEC-PAD). In certain embodiments of all aspects and embodiments, the area-percent of the isoforms in the erythropoietic composition are determined by capillary zone electrophoresis.

In-line pH measuring devices can sometimes be subject to unwanted pH drifts, which may adversely affect process performance, product quality, and product yield in cell culture. Thus, in certain embodiments of all aspects and embodiments, the bioreactor comprises at least two pH measuring devices that are positioned in the bioreactor and configured to be in physical contact with a cultivation medium. In a preferred embodiment of all aspects and embodiments, an alarm is issued when the pH values determined by the at least two pH measuring devices differ by more than 0.05 units from each other. This alarm indicates that the measurements by the probes may no longer be reliable and allows taking appropriate counter measures to avoid any undesirable consequences, such as inadvertently adjusting the cultivation medium pH to the wrong pH set point.

In certain embodiments of all aspects and embodiments, the media specific correlation has been determined by collecting at least one medium-specific data-set for the cultivation medium, wherein the data-set is collected using the steps of
(a) filling the cultivation medium into a tank, preferably a bioreactor, wherein the tank has at least one pH measuring device positioned in the tank and configured to be in physical contact with a cultivation medium,
(b) introducing a gas mixture comprising carbon dioxide into the tank,
(c) measuring the pH in the cultivation medium with the pH measuring device wherein the pH measuring device has been calibrated (under non-sterile conditions) to measure the pH in the cultivation medium,
(d) varying either
   (i) the pH in the cultivation medium and measuring the carbon dioxide concentration in the headspace and/or the exhaust for at least two different pH values, or
   (ii) the carbon dioxide concentration in the gas mixture and measuring the pH values for at least two different carbon dioxide concentrations in the gas mixture, and
(e) obtaining the media specific correlation by mathematically fitting at least two pairs of headspace/exhaust carbon dioxide concentration and corresponding pH value in the cultivation medium.

It should be noted that the collection of the data-set is only possible in a process that is no longer under sterile conditions as the pH probe that is used to measure the pH value of the cultivation medium is calibrated by removing the pH probe from the tank (bioreactor) for calibration, preferably by contacting of the pH measuring device with calibration buffer(s) and putting it back into the tank (bioreactor).

Some time may be needed for an equilibrium to form between the carbon dioxide in the gas phase in the bioreactor and the dissolved carbon dioxide in the cultivation medium. Thus, in certain embodiments of all aspects and embodiments, the carbon dioxide in the headspace and/or the exhaust is determined after an equilibrium has formed between the carbon dioxide in the gas phase in the bioreactor and the dissolved carbon dioxide in the cultivation medium.

The carbon dioxide-based pH calibration method may not only be used in main-stage fermentation, but also during the seed train, i.e. in those cultivation steps that are performed to generate a high enough number of host cells to inoculate the bioreactor of the main stage fermentation. Thus, in certain embodiments of all aspects and embodiments, the recombinant host cells, prior to the inoculating step, have been cultivated in a pre-culture medium in at least one pre-culture step in a bioreactor used for pre-culture, wherein the bioreactor used for pre-culture has at least one pH measuring device positioned in the bioreactor and configured to be in physical contact with the pre-culture medium, and wherein the pH measuring device in the bioreactor used for pre-culture is calibrated according to the following steps:
(i) introducing a gas mixture comprising carbon dioxide gas into the bioreactor
(ii) determining the carbon dioxide concentration in the headspace and/or the exhaust of the bioreactor used for pre-culture,
(iii) calculating the pH of the pre-culture medium based on a media specific correlation, and
(iv) adjusting said pH measuring device to the pH calculated in step (iii).

In certain embodiments of all aspects and embodiments, the method for recombinantly producing a glycoprotein comprises the step of maintaining the pH in the cultivation medium at a desired set point of pH 6.95.

In certain embodiments of all aspects and embodiments, the composition is an erythropoietic composition. In a preferred embodiments of all aspects and embodiments, the glycoprotein is a erythropoiesis-stimulating glycoprotein, in particular erythropoietin.

In certain embodiments of all aspects and embodiments, the erythropoietic composition comprises a defined amount of at least one glycosylated variant of an erythropoiesis-stimulating glycoprotein selected from the group consisting of (a) an erythropoiesis-stimulating glycoprotein having N-Glycans with biantennary structure, (b) an erythropoiesis-stimulating glycoprotein having N-Glycans with triantennary structure, (c) an erythropoiesis-stimulating glycoprotein having N-Glycans with triantennary+1 repeat structure, (d) an erythropoiesis-stimulating glycoprotein having N-Glycans with tetraantennary structure, (e) an erythropoiesis-stimulating glycoprotein having N-Glycans with tetraantennary+1 repeat structure, (f) an erythropoiesis-stimulating glycoprotein having N-Glycans with tetraantennary+2 repeats structure, (g) an erythropoiesis-stimulating glycoprotein having 14 sialic acid residues (Isoform 2), (h) an erythropoiesis-stimulating glycoprotein having 13 sialic acid residues (Isoform 3), (i) an erythropoiesis-stimulating glycoprotein having 12 sialic acid residues (Isoform 4), (j) an erythropoiesis-stimulating glycoprotein having 11 sialic acid residues (Isoform 5), (k) an erythropoiesis-stimulating glycoprotein having 10 sialic acid residues (Isoform 6), (1) an erythropoiesis-stimulating glycoprotein having 9 sialic acid residues (Isoform 7), and (m) an erythropoiesis-stimulating glycoprotein having 8 sialic acid residues (Isoform 8). In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using to a method wherein the measured pH value differs by more than 0.05 units from the pH value in the cultivation medium.

In certain embodiments of all aspects and embodiments, the erythropoietic composition comprises a defined amount of at least one glycosylated variant of an erythropoiesis-stimulating glycoprotein selected from the group consisting of (a) an erythropoiesis-stimulating glycoprotein having N-Glycans with biantennary structure, (b) an erythropoiesis-stimulating glycoprotein having N-Glycans with triantennary structure, (c) an erythropoiesis-stimulating glycoprotein having N-Glycans with triantennary+1 repeat structure, (d) an erythropoiesis-stimulating glycoprotein having N-Glycans with tetraantennary structure, (e) an erythropoiesis-stimulating glycoprotein having N-Glycans with tetraantennary+1 repeat structure, (f) an erythropoiesis-stimulating glycoprotein having N-Glycans with tetraantennary+2 repeats structure. In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium. In certain embodiments of all aspects and embodiments, the erythropoietic composition comprises a defined amount of at least one glycosylated variant of an erythropoiesis-stimulating glycoprotein selected from the group consisting of (a) an erythropoiesis-stimulating glycoprotein having 14 sialic acid residues (Isoform 2), (b) an erythropoiesis-stimulating glycoprotein having 13 sialic acid residues (Isoform 3), (c) an erythropoiesis-stimulating glycoprotein having 12 sialic acid residues (Isoform 4), (d) an erythropoiesis-stimulating glycoprotein having 11 sialic acid residues (Isoform 5), (e) an erythropoiesis-stimulating glycoprotein having 10 sialic acid residues (Isoform 6), (f) an erythropoiesis-stimulating glycoprotein having 9 sialic acid residues (Isoform 7), and (g) an erythropoiesis-stimulating glycoprotein having 8 sialic acid residues (Isoform 8). In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium.

In certain embodiments of all aspects and embodiments, the erythropoietic composition comprises a defined amount of at least one glycosylated variant of an erythropoiesis-stimulating glycoprotein selected from the group consisting of (a) an erythropoiesis-stimulating glycoprotein having N-Glycans with triantennary+1 repeat structure, (b) an erythropoiesis-stimulating glycoprotein having N-Glycans with tetraantennary structure, (c) an erythropoiesis-stimulating glycoprotein having N-Glycans with tetraantennary+1 repeat structure, and (d) an erythropoiesis-stimulating glycoprotein having N-Glycans with tetraantennary+2 repeats structure, (e) an erythropoiesis-stimulating glycoprotein having 14 sialic acid residues (Isoform 2), (f) an erythropoiesis-stimulating glycoprotein having 13 sialic acid residues (Isoform 3), (g) an erythropoiesis-stimulating glycoprotein having 11 sialic acid residues (Isoform 5), (h) an erythropoiesis-stimulating glycoprotein having 10 sialic acid residues (Isoform 6), and (i) an erythropoiesis-stimulating glycoprotein having 9 sialic acid residues (Isoform 7). In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium. In certain embodiments of all aspects and embodiments, the erythropoietic composition comprises a defined amount of at least one glycosylated variant of an erythropoiesis-stimulating glycoprotein selected from the group consisting of (a) an erythropoiesis-stimulating glycoprotein having N-Glycans with triantennary+1 repeat structure, (b) an erythropoiesis-stimulating glycoprotein having N-Glycans with tetraantennary structure, (c) an erythropoiesis-stimulating glycoprotein having N-Glycans with tetraantennary+1 repeat structure, and (d) an erythropoiesis-stimulating glycoprotein having N-Glycans with tetraantennary+2 repeats structure. In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium.

In certain embodiments of all aspects and embodiments, the erythropoietic composition comprises a defined amount of at least one glycosylated variant of an erythropoiesis-stimulating glycoprotein selected from the group consisting of (a) an erythropoiesis-stimulating glycoprotein having 14 sialic acid residues (Isoform 2), (b) an erythropoiesis-stimulating glycoprotein having 13 sialic acid residues (Isoform 3), (c) an erythropoiesis-stimulating glycoprotein having 11 sialic acid residues (Isoform 5), (d) an erythropoiesis-stimulating glycoprotein having 10 sialic acid residues (Isoform 6), and (e) an erythropoiesis-stimulating glycoprotein having 9 sialic acid residues (Isoform 7). In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method where the measured pH value differs by more than 0.05 units from the pH value in the cultivation medium. In a preferred embodiment, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.03 units from the pH value in the cultivation medium.

In certain embodiments of all aspects and embodiments, the erythropoietic composition comprises a defined amount of at least one glycosylated variant of an erythropoiesis-stimulating glycoprotein selected from the group consisting of (a) an erythropoiesis-stimulating glycoprotein having N-Glycans with tetraantennary structure, (b) an erythropoiesis-stimulating glycoprotein having N-Glycans with tetraantennary+1 repeat structure, (c) an erythropoiesis-stimulating glycoprotein having N-Glycans with tetraantennary+2 repeats structure, (d) an erythropoiesis-stimulating glycoprotein having 14 sialic acid residues (Isoform 2), and (e) an erythropoiesis-stimulating glycoprotein having 13 sialic acid residues (Isoform 3). In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium. In certain embodiments of all aspects and embodiments, the erythropoietic composition comprises a defined amount of at least one glycosylated variant of an erythropoiesis-stimulating glycoprotein selected from the group consisting of (a) an erythropoiesis-stimulating glycoprotein having N-Glycans with tetraantennary structure, (b) an erythropoiesis-stimulating glycoprotein having N-Glycans with tetraantennary+1 repeat structure, and (c) an erythropoiesis-stimulating glycoprotein having N-Glycans with tetraantennary+2 repeats structure. In certain embodiments of all aspects and embodiments, the erythropoietic composition comprises a defined amount of at least one glycosylated variant of an erythropoiesis-stimulating glycoprotein selected from the group consisting of (a) an erythropoiesis-stimulating glycoprotein having 14 sialic acid residues (Isoform 2), and (b) an erythropoiesis-stimulating glycoprotein having 13 sialic acid residues (Isoform 3). In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method where the measured pH value differs by more than 0.05 units from the pH value in the cultivation medium. In a preferred embodiment, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method where the measured pH value differs by more than 0.03 units from the pH value in the cultivation medium.

In certain embodiments of all aspects and embodiments, the erythropoietic composition comprises a defined amount of at least one glycosylated variant of an erythropoiesis-stimulating glycoprotein having N-Glycans with tetraantennary structure. In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium.

In certain embodiments of all aspects and embodiments, the erythropoietic composition comprises a defined amount of at least one glycosylated variant of an erythropoiesis-stimulating glycoprotein having N-Glycans with tetraantennary+1 repeat structure. In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium.

In certain embodiments of all aspects and embodiments, the erythropoietic composition comprises a defined amount of at least one glycosylated variant of an erythropoiesis-stimulating glycoprotein having N-Glycans with tetraantennary+2 repeats structure. In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium.

In certain embodiments of all aspects and embodiments, the erythropoietic composition comprises a defined amount of at least one glycosylated variant of an erythropoiesis-stimulating glycoprotein having 14 sialic acid residues (Isoform 2). In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium.

In certain embodiments of all aspects and embodiments, the erythropoietic composition comprises a defined amount of at least one glycosylated variant of an erythropoiesis-stimulating glycoprotein having 13 sialic acid residues (Isoform 3). In certain embodiments of all aspects and embodiments, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method wherein the measured pH value differs by more than 0.05 units from the pH value in the cultivation medium.

In a preferred embodiment, the standard deviation for the relative content of the glycosylated variant calculated for the erythropoietic composition between at least two fermentation batches is decreased, compared to the standard deviation of the relative content of the glycosylated variant calculated for glycoprotein compositions which have been produced using a method where the measured pH value differs by more than 0.03 units from the pH value in the cultivation medium.

In certain embodiments of all aspects and embodiments, the erythropoietic composition comprises one or more selected from the group consisting of: (a) about 3.3 area-% to about 3.8 area-% erythropoiesis-stimulating glycoprotein having N-Glycans with biantennary structure; (b) about 8.6 area-% to about 9.5 area-% erythropoiesis-stimulating glycoprotein having N-Glycans with triantennary structure; (c) about 5.6 area-% to about 5.9 area-% erythropoiesis-stimulating glycoprotein having N-Glycans with triantennary+1 repeat structure; (d) about 42.2 area-% to about 43.4 area-% erythropoiesis-stimulating glycoprotein having N-Glycans with tetraantennary structure; (e) about 27.4 area-% to about 28.1 area-% erythropoiesis-stimulating glycoprotein having N-Glycans with tetraantennary+1 repeat structure; (f) about 10.7 area-% to about 11.6 area-% erythropoiesis-stimulating glycoprotein having N-Glycans with tetraantennary+2 repeat structure, (g) about 13.2 area-% to about 16.0 area-% erythropoiesis-stimulating glycoprotein of Isoform 2; (h) about 24.1 area-% to about 26.5 area-% erythropoiesis-stimulating glycoprotein of Isoform 3; (i) about 23.5 area-% to about 24.6 area-% erythropoiesis-stimulating glycoprotein of Isoform 4; (j) about 17.1 area-% to about 18.6 area-% erythropoiesis-stimulating glycoprotein of Isoform 5; (k) about 9.4 area-% to about 11.8 area-% erythropoiesis-stimulating glycoprotein of Isoform 6; (1) about 3.7 area-% to about 5.5 area-% erythropoiesis-stimulating glycoprotein of Isoform 7; and (m) about 0.9 area-% to about 1.6 area-% erythropoiesis-stimulating glycoprotein of Isoform 8. In certain embodiments of all aspects and embodiments, the area-percent of N-Glycans of the erythropoiesis-stimulating glycoprotein are determined by anion exchange chromatography after enzymatic release of N-glycosidically-linked oligosaccharides from the glycoprotein and desialylation. In certain embodiments the anion exchange chromatography is high performance anion-exchange chromatography with pulsed amperometric detection (HPAEC-PAD). In certain embodiments of all aspects and embodiments, the area-percent of the isoforms in the erythropoietic composition are determined by capillary zone electrophoresis.

In certain embodiments of all aspects and embodiments, the erythropoietic composition comprises one or more selected from the group consisting of: (a) about 3.3 area-% to about 3.8 area-% erythropoiesis-stimulating glycoprotein having N-Glycans with biantennary structure; (b) about 8.6 area-% to about 9.5 area-% erythropoiesis-stimulating glycoprotein having N-Glycans with triantennary structure; (c) about 5.6 area-% to about 5.9 area-% erythropoiesis-stimulating glycoprotein having N-Glycans with triantennary+1 repeat structure; (d) about 42.2 area-% to about 43.4 area-% erythropoiesis-stimulating glycoprotein having N-Glycans with tetraantennary structure; (e) about 27.4 area-% to about 28.1 area-% erythropoiesis-stimulating glycoprotein having N-Glycans with tetraantennary+1 repeat structure; and (f) about 10.7 area-% to about 11.6 area-% erythropoiesis-stimulating glycoprotein having N-Glycans with tetraantennary+2 repeat structure, preferably as determined by anion exchange chromatography after enzymatic release of N-glycosidically-linked oligosaccharides from the glycoprotein and desialylation. In certain embodiments of all aspects and embodiments, the erythropoietic composition comprises one or more selected from the group consisting of: (a) about 13.2 area-% to about 16.0 area-% erythropoiesis-stimulating glycoprotein of Isoform 2; (b) about 24.1 area-% to about 26.5 area-% erythropoiesis-stimulating glycoprotein of Isoform 3; (c) about 23.5 area-% to about 24.6 area-% erythropoiesis-stimulating glycoprotein of Isoform 4; (j) about 17.1 area-% to about 18.6 area-% erythropoiesis-stimulating glycoprotein of Isoform 5; (k) about 9.4 area-% to about 11.8 area-% erythropoiesis-stimulating glycoprotein of Isoform 6; (e) about 3.7 area-% to about 5.5 area-% erythropoiesis-stimulating glycoprotein of Isoform 7; and (f) about 0.9 area-% to about 1.6 area-% erythropoiesis-stimulating glycoprotein of Isoform 8, preferably as determined by capillary zone electrophoresis.

In certain embodiments of all aspects and embodiments, the erythropoietic composition comprises one or more selected from the group consisting of: (a) about 8.6 area-% to about 9.5 area-% erythropoiesis-stimulating glycoprotein having N-Glycans with triantennary structure; (b) about 5.6 area-% to about 5.9 area-% erythropoiesis-stimulating glycoprotein having N-Glycans with triantennary+1 repeat structure; (c) about 42.2 area-% to about 43.4 area-% erythropoiesis-stimulating glycoprotein having N-Glycans with tetraantennary structure; (d) about 27.4 area-% to about 28.1 area-% erythropoiesis-stimulating glycoprotein having N-Glycans with tetraantennary+1 repeat structure; preferably as determined by anion exchange chromatography after enzymatic release of N-glycosidically-linked oligosaccharides from the glycoprotein and desialylation; (e) about 13.2 area-% to about 16.0 area-% erythropoiesis-stimulating glycoprotein of Isoform 2; (f) about 24.1 area-% to about 26.5 area-% erythropoiesis-stimulating glycoprotein of Isoform 3; (g) about 17.1 area-% to about 18.6 area-% erythropoiesis-stimulating glycoprotein of Isoform 5; (h) about 9.4 area-% to about 11.8 area-% erythropoiesis-stimulating glycoprotein of Isoform 6; and (i) about 3.7 area-% to about 5.5 area-% erythropoiesis-stimulating glycoprotein of Isoform 7; preferably as determined by capillary zone electrophoresis.

In certain embodiments of all aspects and embodiments, the erythropoietic composition comprises one or more selected from the group consisting of: (a) about 8.6 area-% to about 9.5 area-% erythropoiesis-stimulating glycoprotein having N-Glycans with triantennary structure; (b) about 5.6 area-% to about 5.9 area-% erythropoiesis-stimulating glycoprotein having N-Glycans with triantennary+1 repeat structure; (c) about 42.2 area-% to about 43.4 area-% erythropoiesis-stimulating glycoprotein having N-Glycans with tetraantennary structure; and (d) about 27.4 area-% to about 28.1 area-% erythropoiesis-stimulating glycoprotein having N-Glycans with tetraantennary+1 repeat structure, preferably as determined by anion exchange chromatography after enzymatic release of N-glycosidically-linked oligosaccharides from the glycoprotein and desialylation. In certain embodiments of all aspects and embodiments, the erythropoietic composition comprises one or more selected from the group consisting of: (a) about 13.2 area-% to about 16.0 area-% erythropoiesis-stimulating glycoprotein of Isoform 2; (b) about 24.1 area-% to about 26.5 area-% erythropoiesis-stimulating glycoprotein of Isoform 3; (c) about 17.1 area-% to about 18.6 area-% erythropoiesis-stimulating glycoprotein of Isoform 5; (d) about 9.4 area-% to about 11.8 area-% erythropoiesis-stimulating glycoprotein of Isoform 6; and (e) about 3.7 area-% to about 5.5 area-% erythropoiesis-stimulating glycoprotein of Isoform 7, preferably as determined by capillary zone electrophoresis.

In certain embodiments of all aspects and embodiments, the erythropoietic composition comprises one or more selected from the group consisting of: (a) about 42.2 area-% to about 43.4 area-% erythropoiesis-stimulating glycoprotein having N-Glycans with tetraantennary structure; (b) about 27.4 area-% to about 28.1 area-% erythropoiesis-stimulating glycoprotein having N-Glycans with tetraantennary+1 repeat structure; (c) about 10.7 area-% to about 11.6 area-% erythropoiesis-stimulating glycoprotein having N-Glycans with tetraantennary+2 repeat structure, preferably as determined by anion exchange chromatography after enzymatic release of N-glycosidically-linked oligosaccharides from the glycoprotein and desialylation; and (d) about 13.2 area-% to about 16.0 area-% erythropoiesis-stimulating glycoprotein of Isoform 2; and (e) about 24.1 area-% to about 26.5 area-% erythropoiesis-stimulating glycoprotein of Isoform 3; preferably as determined by capillary zone electrophoresis.

In certain embodiments of all aspects and embodiments, the erythropoietic composition comprises one or more selected from the group consisting of: (a) about 42.2 area-% to about 43.4 area-% erythropoiesis-stimulating glycoprotein having N-Glycans with tetraantennary structure; (b) about 27.4 area-% to about 28.1 area-% erythropoiesis-stimulating glycoprotein having N-Glycans with tetraantennary+1 repeat structure; and (c) about 10.7 area-% to about 11.6 area-% erythropoiesis-stimulating glycoprotein having N-Glycans with tetraantennary+2 repeat structure, preferably as determined by anion exchange chromatography after enzymatic release of N-glycosidically-linked oligosaccharides from the glycoprotein and desialylation. In certain embodiments of all aspects and embodiments, the erythropoietic composition comprises one or more selected from the group consisting of: (a) about 13.2 area-% to about 16.0 area-% erythropoiesis-stimulating glycoprotein of Isoform 2; and (b) about 24.1 area-% to about 26.5 area-% erythropoiesis-stimulating glycoprotein of Isoform 3, preferably as determined by capillary zone electrophoresis.

In certain embodiments of all aspects and embodiments, the erythropoietic composition comprises one or more selected from the group consisting of (a) about 42.2 area-% to about 43.4 area-% erythropoiesis-stimulating glycoprotein having N-Glycans with tetraantennary structure; (b) about 27.4 area-% to about 28.1 area-% erythropoiesis-stimulating glycoprotein having N-Glycans with tetraantennary+1 repeat structure; and (c) about 10.7 area-% to about 11.6 area-% of the N-Glycans of the erythropoiesis-stimulating glycoprotein have a tetraantennary+2 repeat structure, preferably as determined by anion exchange chromatography after enzymatic release of N-glycosidically-linked oligosaccharides from the glycoprotein and desialylation.

In certain embodiments of all aspects and embodiments, the erythropoietic composition comprises from about 26.5 area-% to about 28.2 area-% erythropoiesis-stimulating glycoprotein having N-Glycans with tetraantennary+1 repeat structure, preferably as determined by anion exchange chromatography after enzymatic release of N-glycosidically-linked oligosaccharides from the glycoprotein and desialylation. In certain embodiments of all aspects and embodiments, the erythropoietic composition comprises from about 27.0 area-% to about 28.1 area-% erythropoiesis-stimulating glycoprotein having N-Glycans with tetraantennary+1 repeat structure, preferably as determined by anion exchange chromatography after enzymatic release of N-glycosidically-linked oligosaccharides from the glycoprotein and desialylation. In certain embodiments of all aspects and embodiments, the erythropoietic composition comprises from about 27.4 area-% to about 28.1 area-% erythropoiesis-stimulating glycoprotein having N-Glycans with tetraantennary+1 repeat structure, preferably as determined by anion exchange chromatography after enzymatic release of N-glycosidically-linked oligosaccharides from the glycoprotein and desialylation.

In certain embodiments of all aspects and embodiments, the erythropoietic composition comprises from about 42.2 area-% to about 43.4 area-% erythropoiesis-stimulating glycoprotein having N-Glycans with tetraantennary structure, preferably as determined by anion exchange chromatography after enzymatic release of N-glycosidically-linked oligosaccharides from the glycoprotein and desialylation.

In certain embodiments of all aspects and embodiments, the erythropoietic composition comprises from about 10.7 area-% to about 11.6 area-% erythropoiesis-stimulating glycoprotein having N-Glycans with tetraantennary+2 repeat structure, preferably as determined by anion exchange chromatography after enzymatic release of N-glycosidically-linked oligosaccharides from the glycoprotein and desialylation.

In certain embodiments of all aspects and embodiments, the erythropoietic composition has an average sialic acid content from about 10.5 to 11.0 moles of sialic acid per mole of the erythropoiesis-stimulating glycoprotein.

In a preferred embodiment of all aspects and embodiments, the erythropoiesis-stimulating glycoprotein is erythropoietin.

Disclosed herein are also glycoproteins, in particular erythropoiesis-stimulating glycoproteins, particularly erythropoietin, that are obtained by the methods discloses herein. Further disclosed are glycoproteins, in particular erythropoiesis-stimulating glycoproteins, particularly erythropoietin, that are obtainable by the methods disclosed herein.

A recombinant glycoprotein disclosed herein, in particular erythropoiesis-stimulating glycoproteins, particularly erythropoietin, may be incorporated (e.g., formulated) into a pharmaceutical composition. Accordingly, in another aspect, the present invention relates to a method for producing a pharmaceutical composition comprising a glycoprotein having a reduced batch-to-batch variability with regard to one or more glycosylated variants, wherein said method comprises carrying out the method for producing a glycoprotein composition and/or for reducing the variability of glycosylation variants between batches of said recombinant glycoprotein as described herein. One aspect are also pharmaceutical compositions prepared with a glycoprotein disclosed herein.

In some embodiments, the method for producing a pharmaceutical composition further comprises combining said recombinant glycoprotein (i.e., obtained from the methods described herein) together with a pharmaceutically acceptable carrier. Such a pharmaceutical composition is useful as an alternative and/or improved composition for the prevention and/or treatment of one or more diseases relative to a corresponding reference glycoprotein. Pharmaceutical compositions comprising a glycoprotein can be formulated by methods known to those skilled in the art. The pharmaceutical composition can be administered parenterally in the form of an injectable formulation comprising a sterile solution or suspension in water or another pharmaceutically acceptable liquid. For example, the pharmaceutical composition may be formulated by suitably combining the glycoprotein with pharmaceutically acceptable vehicles or media, such as sterile water, physiological saline, vegetable oil, emulsifier, suspension agent, surfactant, stabilizer, flavoring excipient, diluent, vehicle, preservative, binder. The amount of active ingredient included in the pharmaceutical preparations is such that a suitable dose within the designated range is provided.

A sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, and the like.

Other items that may be included are a buffer such as a phosphate buffer, or sodium acetate buffer, a soothing agent such as procaine hydrochloride, a stabilizer such as benzyl alcohol or phenol, and an antioxidant. A formulated injection can be packaged in a suitable ampule. Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection. A suitable means of administration can be selected based on the age and condition of the patient. A single dose of the pharmaceutical composition containing a modified glycoprotein can be selected from a range of 0.001 to 1000 mg/kg of body weight. On the other hand, a dose can be selected in the range of 0.001 to 100000 mg/body weight, but the present disclosure is not limited to such ranges. The dose and method of administration varies depending on the weight, age, condition, and the like of the patient, and can be suitably selected as needed by those skilled in the art.

Other technical features may be readily apparent to one skilled in the art from the following figures, descriptions, and claims.

### 3. Analytical Assays

Methods for analyzing and quantifying glycosylated variants of glycoprotein are well known to the person skilled in the art.

The glycosylated variants may be determined directly from the cultivation medium or the cell culture supernatant, that is, from the cultivation medium once the host cells have been removed. A glycoprotein composition may be subjected to one or more purification steps before determination of glycosylated variants by methods known to the person skilled in the art, such as affinity chromatography (in case of antibodies, protein A chromatography), ion exchange chromatography, hydrophobic interaction chromatography, ultrafiltration, diafiltration, precipitation, centrifugation, and/or depth filtration. The person skilled in art is aware of methods that are suitable and useful for the preparation of glycoprotein samples for analytic assays. In certain embodiments of all aspects and embodiments, quantitation of a glycosylated variant is based on the relative area percent of a detected peak that corresponds to the glycosylated variant. The relative area percent of a peak corresponds to the integrated area under a peak in a chromatogram as measured by a detector, relative to the total integrated peak area of the entire chromatogram, for example of a chromatogram generated by high performance anion-exchange chromatography with pulsed amperometric detection or by capillary zone electrophoresis. In certain embodiments of all aspects and embodiments, the glycoprotein composition obtained by cultivating a recombinant host cell has been subjected to further isolation and/or purification steps before qualitatively and/or quantitatively analyzing the glycosylated variants. In one embodiment, the glycoprotein is erythropoietin and has been purified using a downstream purification process comprising one or more steps selected from the group consisting of capture chromatography (e.g. blue sepharose chromatography), hydrophobic interaction chromatography (e.g. butyl toyopearl chromatography), adsorption chromatography (e.g. hydroxyapatite ultragel chromatography), preparative reversed-phase high-performance liquid chromatography (RP-HPLC) and anion exchange chromatography (e.g. DEAE Sepharose chromatography). The process may further comprise a virus removal step (e.g. nanofiltration).

In certain embodiments of all aspects and embodiments, quantitation of a glycosylated variant of a glycoprotein characterized by a certain N-glycan structure is based on the relative area percent of a detected peak corresponding to the glycosylated variant, as determined by anion exchange chromatography after enzymatic release of N-glycosidically-linked oligosaccharides from the glycoprotein using the enzyme N-glycosidase F and optionally enzymatic desialylation. In certain embodiments, the anion exchange chromatography is a high performance anion-exchange chromatography with pulsed amperometric detection (HPAEC-PAD). In certain embodiments of all aspects and embodiments, the quantitation of sialylated isoforms of a glycoprotein is based on the relative area percent of a detected peak that corresponds to the glycosylated variant, as determined by capillary zone electrophoresis.

Erythropoiesis-stimulating glycoproteins disclosed herein may be identified, screened for, or characterized for their physical/chemical properties and/or biological activities by various assays known in the art. In certain embodiments of all aspects and embodiments, quantitation of a glycosylated variant of an erythropoiesis-stimulating glycoprotein, such as erythropoietin, that is characterized by a certain N-glycan structure is based on the relative area percent of a detected peak that corresponds to the glycosylated variant, as determined by anion exchange chromatography after enzymatic release of N-glycosidically-linked oligosaccharides from the glycoprotein, preferably using the enzyme N-glycosidase F, and desialylation, preferably by using the enzyme neuraminidase. In certain embodiments, the anion exchange chromatography is high performance anion-exchange chromatography with pulsed amperometric detection (HPAEC-PAD). In certain embodiments, the quantitation is performed using the analytical assay of the erythropoietin N-glycan profile described herein. In certain embodiments of all aspects and embodiments, quantitation of sialylated isoforms of an erythropoiesis stimulating glycoprotein is based on the relative area percent of a detected peak corresponding to the glycosylated variant, as determined by capillary zone electrophoresis. In certain embodiments of all aspects and embodiments, quantitation of sialylated isoforms of an erythropoiesis stimulating glycoprotein is performed using the analytical assay of for determining Erythropoiesis-stimulating glycoprotein isoform content described herein.

For instance, the European Pharmacopoeia describes assays for N-Glycan analysis of Erythropoietin and for Capillary zone electrophoresis (CZE) of Erythropoietin isoforms (Ph. Eur. 11.0, 2673 (01/2023)). A method for CZE of Erythropoietin isoforms is for instance also described in Brinks et al. (Pharm Res (2011) 28:386-393; DOI 10.1007/s11095-010-0288-2) or in Zhang et al. (J Pharm Biomed Anal 50(3):538-43; DOI: 10.1016/j.jpba.2009.05.007). In certain embodiments of all aspects and embodiments, the relative content of erythropoietin N-glycans is determined according to the methods described in the European pharmacopeia (Ph. Eur. 11.0, 2673 [01/2023]). In certain embodiments of all aspects and embodiments, the relative content of erythropoietin isoforms is determined according to the methods described in the European pharmacopeia (Ph. Eur. 11.0, 2673 [01/2023]). In the following, exemplary methods for analyzing erythropoiesis-stimulating glycoproteins will be described.

### Assay for Determining Erythropoiesis-Stimulating Glycoprotein Isoform Content

The isoform distribution may be determined by means of capillary zone electrophoresis. In this method, separation is carried out in a constant field strength in an uncoated glass capillary with a uniform buffer system, the pH of which lies above the pI-values of the EPO isoforms. Since all EPO isoforms are negatively charged and are transported by the endoosmotic flow towards the cathode, the basic isoforms are detected first. Samples are diafiltered in water. The capillary is rinsed with an electrolyte solution followed by the application of the diluted samples into the capillary. The separation takes place by applying a high voltage of 25,000 V. A mobile buffer is used that has an excess of positive ions which result in an electroosmotic flow. The proteins in the capillary may be detected via photometric detection using a quartz capillary and a diode array detector. The isoforms may be determined quantitatively by integrating the peaks corresponding to Isoform 1 to Isoform 9 to obtain the area under the peak [relative area-%].

### Assay for Determining Erythropoiesis-Stimulating Glycoprotein N-Glycan Profile

The relative distribution of the N-glycans may be determined using an enzymatic test procedure in combination with a High-Performance Anion-Exchange Chromatography system with Pulsed Amperometric Detection (HPAEC-PAD), essentially as described in WO 99/28346. In this procedure, the N-glycosidically-linked oligosaccharides of erythropoietin are initially cleaved by the enzyme N-glycosidase F. In addition, the terminal sialic acids are removed from the oligosaccharides with the aid of the enzyme neuraminidase. After removal of the protein fraction via ultrafiltration, the N-oligosaccharides that are obtained are separated and analyzed with the help of the HPAEC-PAD and a suitable data recording system. An erythropoietin reference standard which is treated in exactly the same manner as the samples is measured during the series of tests. The area-percentage of each glycosylated variant in the preparation may be calculated from the corresponding peak in the chromatogram of the glycans produced (e.g. biantennary, triantennary, triantennary+1 repeat, tetraantennary, tetraantennary+1 repeat, tetraantennary+2 repeats.

### Determination of the Content of Sialic Acid Residues

The sialic acid content may be determined chromatographically by means of HPAEC-PAD after enzymatic cleavage of the sialic acids with neuraminidase, essentially as described in WO 99/28346. For this, erythropoietin samples are diluted in a sodium phosphate buffer, pH 7.2. Half of each preparation is used to determine the exact erythropoietin amount in the samples by means of RP-HPLC. Neuraminidase is added to the second half of each preparation and incubated overnight at 37° C. Subsequently, the digestion mixtures are split in half, diluted with water and 50 μl thereof are applied to the HPAEC-PAD. The amount of sialic acids in the applied sample may be determined with the help of a calibration line which may be obtained from values of a sialic acid standard that is analyzed in the same test run. The sialic acid content (mole sialic acid/mole erythropoietin) may be calculated from the result of the sialic acid determination and the determination of the amount of erythropoietin used by means of RP-HPLC.

### 4. Embodiments of the Invention

The current invention encompasses at least the following independent aspects and dependent embodiments:
1. A method for recombinantly producing a glycoprotein composition comprising at least one glycoprotein, wherein the method comprises the step of
cultivating a recombinant host cell which expresses the glycoprotein in a cultivation medium in a bioreactor having a pH measuring device positioned in the bioreactor and configured to be in physical contact with the cultivation medium,
characterized in that
(a) the cultivating is performed under sterile conditions,
(b) the pH value measured with the pH measuring device differs by 0.05 units or less from the pH value of the cultivation medium, and
(c) the relative content of at least one glycosylated variant in the glycoprotein composition has reduced batch-to-batch variability, compared to a method where the pH value measured by the pH measuring device differs by more than 0.05 units, preferably by more than 0.03 units, from the pH value in the cultivation medium,
and thereby producing the glycoprotein composition.
2. A method for recombinantly producing a glycoprotein composition comprising at least one glycoprotein in a recombinant host cell which expresses the glycoprotein, comprising the steps of
(a) providing a bioreactor comprising a pH measuring device positioned in the bioreactor and configured to be in physical contact with a cultivation medium,
(b) closing and sterilizing the bioreactor,
(c) filling the cultivation medium into the bioreactor,
(d) calibrating the pH measuring device,
(e) inoculating the bioreactor with the recombinant host cell,
(f) cultivating the recombinant host cell under conditions suitable for producing the glycoprotein composition, and
(g) thereby producing the glycoprotein composition,
characterized in that the calibrating of step (d) comprises the steps of
(i) introducing a gas mixture comprising carbon dioxide gas into the bioreactor,
(ii) determining the carbon dioxide concentration in the headspace and/or the exhaust,
(iii) calculating the pH value of the cultivation medium based on a media specific correlation, and
(iv) adjusting said pH measuring device to the pH value calculated in step (iii).
3. The method of embodiment 2, characterized in that after the calibration step the pH measured with the pH measuring device differs by 0.05 units or less, preferably by 0.03 units or less, from the pH value of the cultivation medium.
4. The method of any one of embodiments 1 to 3, characterized in that the glycoprotein composition comprises at least one glycosylated variant of the glycoprotein, and
that the standard deviation of the relative content of the glycosylated variant calculated for glycoprotein compositions from at least two fermentation batches is decreased, compared to the standard deviation of the relative content of the glycosylated variant calculated for glycoprotein compositions which have been produced using a method wherein the pH value measured by the pH measuring device differs by more than 0.05 units, preferably by more than 0.03 units, from the pH value in the cultivation medium.
5. The method of embodiment 4, wherein the standard deviation is calculated for glycoprotein compositions from at least five, preferably at least ten, fermentation batches.
6. The method of any one of embodiments 1 to 5, wherein the recombinant host cell is a mammalian cell.
7. The method of any one of embodiments 1 to 6, wherein the recombinant host cell is a CHO cell.
8. The method of any one of embodiments 4 to 7, wherein the glycosylated variant is selected from the group consisting of N-glycan variants, O-glycan variants, sialylation variants, mannosylation variants, galactosylation variants and fucosylation variants.
9. The method of any one of embodiments 1 to 8, wherein the cultivation medium comprises a carbonate buffer.
10. The method of any one of embodiments 1 to 9, comprising the step of maintaining the pH in the cultivation medium at a desired set point of pH 6.95.
11. The method of any one of embodiments 1 to 10, wherein the desired pH set point in the cultivation medium is adjusted by
(a) increasing carbon dioxide influx if pH is above the desired set point, or
(b) adding an alkaline component, preferably 1 mol/L NaOH, to the cultivation medium, if the pH is below the desired set point.
12. The method of any one of embodiments 1 to 11, wherein the carbon dioxide is introduced into the bioreactor by sparging.
13. The method of any one of embodiments 1 to 12, wherein the bioreactor comprises at least two pH measuring devices positioned in the bioreactor and configured to be in physical contact with a cultivation medium.
14. The method of any one of embodiments 1 to 13, wherein an alarm is issued when the pH values determined by the at least two pH measuring devices differ by more than 0.05 units from each other.
15. The method of any one of embodiments 1 to 14, wherein the media specific correlation has been determined by collecting at least one medium-specific data-set for the cultivation medium, wherein the data-set is collected using the steps of
(a) filling the cultivation medium into a tank, preferably a bioreactor, wherein the tank has at least one pH measuring device positioned in the tank and configured to be in physical contact with a cultivation medium,
(b) introducing a gas mixture comprising carbon dioxide into the tank,
(c) measuring the pH in the cultivation medium with the pH measuring device wherein the pH measuring device has been calibrated under non-sterile conditions to measure the pH in the cultivation medium,
(d) varying either
(i) the pH in the cultivation medium and measuring carbon dioxide concentration in the headspace and/or exhaust of the tank for at least two different pH values, or
(ii) the carbon dioxide concentration in the gas mixture and measuring the pH values for at least two different carbon dioxide concentrations in the gas mixture, and
(e) obtaining the media specific correlation by mathematically fitting at least two pairs of headspace and/or exhaust carbon dioxide concentration and corresponding pH in the cultivation medium.
16. The method of any one of embodiments 1 to 15, wherein the carbon dioxide concentration in the headspace and/or exhaust is determined after an equilibrium has formed between the carbon dioxide in the gas phase in the tank, preferably the bioreactor, and the dissolved carbon dioxide in the cultivation medium.
17. The method of any one of embodiments 1 to 16, characterized in that the recombinant host cells prior to inoculation have been cultivated in a pre-culture medium in at least one pre-culture step in a bioreactor used for pre-culture, wherein the bioreactor used for pre-culture has at least one pH measuring device positioned in the bioreactor and configured to be in physical contact with a pre-culture medium, and wherein the pH measuring device in the bioreactor used for pre-culture is calibrated according to steps (i) to (iii) of embodiment 2.
18. The method of any one of embodiments 1 to 17, whereby the glycoprotein composition is an erythropoietic composition.
19. The method of any one of embodiments 1 to 18, wherein the glycoprotein is an erythropoiesis-stimulating glycoprotein, preferably erythropoietin.
20. The method of embodiment 18 or 19, further characterized in that the erythropoietic composition comprises a defined amount of at least one glycosylated variant of an erythropoiesis-stimulating glycoprotein selected from the group consisting of
(a) an erythropoiesis-stimulating glycoprotein having N-Glycans with biantennary structure;
(b) an erythropoiesis-stimulating glycoprotein having N-Glycans with triantennary structure;
(c) an erythropoiesis-stimulating glycoprotein having N-Glycans with triantennary+1 repeat structure;
(d) an erythropoiesis-stimulating glycoprotein having N-Glycans with tetraantennary structure;
(e) an erythropoiesis-stimulating glycoprotein having N-Glycans with tetraantennary+1 repeat structure;
(f) an erythropoiesis-stimulating glycoprotein having N-Glycans with tetraantennary+2 repeats structure;
(g) an erythropoiesis-stimulating glycoprotein having 14 sialic acid residues (Isoform 2);
(h) an erythropoiesis-stimulating glycoprotein having 13 sialic acid residues (Isoform 3)
(i) an erythropoiesis-stimulating glycoprotein having 12 sialic acid residues (Isoform 4);
(j) an erythropoiesis-stimulating glycoprotein having 11 sialic acid residues (Isoform 5);
(k) an erythropoiesis-stimulating glycoprotein having 10 sialic acid residues (Isoform 6);
(l) an erythropoiesis-stimulating glycoprotein having 9 sialic acid residues (Isoform 7); and
(m) an erythropoiesis-stimulating glycoprotein having 8 sialic acid residues (Isoform 8).
21. The method of any one of embodiments 18 to 20, characterized in that the erythropoietic composition comprises a defined amount of at least one glycosylated variant of an erythropoiesis-stimulating glycoprotein selected from the group consisting of
(a) an erythropoiesis-stimulating glycoprotein having N-Glycans with tetraantennary structure;
(b) an erythropoiesis-stimulating glycoprotein having N-Glycans with tetraantennary+1 repeat structure;
(c) an erythropoiesis-stimulating glycoprotein having N-Glycans with tetraantennary+2 repeats structure;
(d) an erythropoiesis-stimulating glycoprotein having 14 sialic acid residues (Isoform 2); and
(e) an erythropoiesis-stimulating glycoprotein having 13 sialic acid residues (Isoform 3).
22. The method of any one of embodiments 18 to 21, characterized in that the erythropoietic composition comprises one or more selected from the group consisting of:
(a) about 3.3 area-% to about 3.8 area-% erythropoiesis-stimulating glycoprotein having N-Glycans with biantennary structure;
(b) about 8.6 area-% to about 9.5 area-% erythropoiesis-stimulating glycoprotein having N-Glycans with triantennary structure;
(c) about 5.6 area-% to about 5.9 area-% erythropoiesis-stimulating glycoprotein having N-Glycans with triantennary+1 repeat structure;
(d) about 42.2 area-% to about 43.4 area-% erythropoiesis-stimulating glycoprotein having N-Glycans with tetraantennary structure;
(e) about 27.4 area-% to about 28.1 area-% erythropoiesis-stimulating glycoprotein having N-Glycans with tetraantennary+1 repeat structure;
(f) about 10.7 area-% to about 11.6 area-% erythropoiesis-stimulating glycoprotein having N-Glycans with tetraantennary+2 repeat structure;
(g) about 13.2 area-% to about 16.0 area-% erythropoiesis-stimulating glycoprotein of Isoform 2;
(h) about 24.1 area-% to about 26.5 area-% erythropoiesis-stimulating glycoprotein of Isoform 3;
(i) about 23.5 area-% to about 24.6 area-% erythropoiesis-stimulating glycoprotein of Isoform 4;
(j) about 17.1 area-% to about 18.6 area-% erythropoiesis-stimulating glycoprotein of Isoform 5;
(k) about 9.4 area-% to about 11.8 area-% erythropoiesis-stimulating glycoprotein of Isoform 6;
(l) about 3.7 area-% to about 5.5 area-% erythropoiesis-stimulating glycoprotein of Isoform 7; and/or
(m) about 0.9 area-% to about 1.6 area-% erythropoiesis-stimulating glycoprotein of Isoform 8,
preferably wherein (a) to (f) are determined by anion exchange chromatography after enzymatic release of N-glycosidically-linked oligosaccharides from the glycoprotein and desialylation and (g) to (m) are determined by capillary zone electrophoresis.
23. The method of any one of embodiments 18 to 22, characterized in that the erythropoietic composition comprises a defined amount of at least one glycosylated variant of an erythropoiesis-stimulating glycoprotein selected from the group consisting of:
(a) about 42.2 area-% to about 43.4 area-% erythropoiesis-stimulating glycoprotein having N-Glycans with tetraantennary structure;
(b) about 27.4 area-% to about 28.1 area-% erythropoiesis-stimulating glycoprotein having N-Glycans with tetraantennary+1 repeat structure;
(c) about 10.7 area-% to about 11.6 area-% erythropoiesis-stimulating glycoprotein having N-Glycans with tetraantennary+2 repeat structure;
(d) about 13.2 area-% to about 16.0 area-% erythropoiesis-stimulating glycoprotein of Isoform 2; and
(e) about 24.1 area-% to about 26.5 area-% erythropoiesis-stimulating glycoprotein of Isoform 3,
preferably wherein (a) to (c) are determined by anion exchange chromatography after enzymatic release of N-glycosidically-linked oligosaccharides from the glycoprotein and desialylation and (d) to (e) are determined by capillary zone electrophoresis.
24. The method of any one of embodiments 18 to 23, characterized in that the erythropoietic composition comprises a defined amount of at least one glycosylated variant of an erythropoiesis-stimulating glycoprotein selected from the group consisting of:
(a) about 42.2 area-% to about 43.4 area-% erythropoiesis-stimulating glycoprotein having N-Glycans with tetraantennary structure;
(b) about 27.4 area-% to about 28.1 area-% erythropoiesis-stimulating glycoprotein having N-Glycans with tetraantennary+1 repeat structure; and
(c) about 10.7 area-% to about 11.6 area-% erythropoiesis-stimulating glycoprotein having N-Glycans with a tetraantennary+2 repeat structure,
preferably as determined by anion exchange chromatography after enzymatic release of N-glycosidically-linked oligosaccharides from the glycoprotein and desialylation.
25. The method of any one of embodiments 18 to 24, characterized in that the erythropoietic composition comprises about 27.4 area-% to about 28.1 area-% erythropoiesis-stimulating glycoprotein having N-Glycans with tetraantennary+1 repeat structure, preferably as determined by anion exchange chromatography after enzymatic release of N-glycosidically-linked oligosaccharides from the glycoprotein and desialylation.
26. The method of any one of embodiments 18 to 25, wherein the erythropoietic composition has an average sialic acid content from about 10.5 to 11.0 moles of sialic acid per mole of the erythropoiesis-stimulating glycoprotein.
27. The method of any one of embodiments 18 to 26, wherein the erythropoiesis-stimulating glycoprotein is erythropoietin.
28. A method of reducing the variability of the relative content of at least one glycosylated variant between batches of a recombinant glycoprotein composition, the method comprising:
(a) providing a bioreactor comprising a pH measuring device positioned in the bioreactor and configured to be in physical contact with a cultivation medium,
(b) closing and sterilizing the bioreactor,
(c) filling the cultivation medium into the bioreactor,
(d) calibrating the pH measuring device,
(e) inoculating the bioreactor with a recombinant host cell which expresses the glycoprotein, and
(f) cultivating the recombinant host cell under conditions suitable for producing the glycoprotein composition, and
(g) thereby producing the glycoprotein composition,
wherein the calibrating of step (d) comprises the steps of
(i) introducing a gas mixture comprising carbon dioxide gas into the bioreactor,
(ii) determining the carbon dioxide concentration in the headspace and/or the exhaust of the bioreactor,
(iii) calculating the pH value of the cultivation medium based on a media specific correlation, and
(iv) adjusting said pH measuring device to the pH value calculated in step iii), and
wherein steps (a) to (g) result in a first batch of said recombinant glycoprotein with a defined relative content of the glycosylated variant;
(h) repeating the steps (a) to (g) resulting in at least one subsequent batch of said glycoprotein, wherein the relative content of the at least one glycosylated variant of said first and at least one subsequent batch has reduced batch-to-batch variability.
29. The method of embodiment 28, wherein the standard deviation of the relative content of the glycoprotein calculated for said first and at least one subsequent batch is 1% or less, particularly 0.8% or less, most particularly 0.5% or less of the median of the pH value.
30. Use of a carbon-dioxide based method of calibrating a pH measurement device positioned in a bioreactor and configured to be in physical contact with a cultivation medium in the bioreactor for reducing the standard deviation of the relative content of glycosylated variants in a glycoprotein composition calculated between at least two fermentation batches, compared to a glycoprotein composition produced using a pH measurement method wherein the measured pH value differs by more than 0.05 units, preferably by more than 0.03 units, from the pH value in the cultivation medium.
31. The use of embodiment 30, wherein the carbon-dioxide based method of calibrating a pH measurement device comprises the steps of
(i) introducing a gas mixture comprising carbon dioxide gas into the bioreactor comprising the cultivation medium,
(ii) determining the carbon dioxide concentration in the headspace and/or the exhaust of the bioreactor,
(iii calculating the pH value of the cultivation medium based on a Media specific correlation, and
(iv) adjusting the pH measuring device to the pH value calculated in step (iii).
32. The use of embodiment 30 or 31, wherein the carbon dioxide is introduced into the bioreactor by sparging.
33. The use of any one of embodiments 30 to 32, wherein the cultivation medium comprises a carbonate-buffer.
34. The use of any one of embodiments 30 to 33, wherein the glycoprotein composition is an erythropoietic composition.
35. The use of embodiment 34, wherein the glycoprotein is an erythropoiesis-stimulating glycoprotein.
36. The use of embodiment 34 or 35, wherein the erythropoietic composition comprises a defined amount of at least one glycosylated variant of an erythropoiesis-stimulating glycoprotein selected from the group consisting of
(a) an erythropoiesis-stimulating glycoprotein having N-Glycans with biantennary structure;
(b) an erythropoiesis-stimulating glycoprotein having N-Glycans with triantennary structure;
(c) an erythropoiesis-stimulating glycoprotein having N-Glycans with triantennary+1 repeat structure;
(d) an erythropoiesis-stimulating glycoprotein having N-Glycans with tetraantennary structure;
(e) an erythropoiesis-stimulating glycoprotein having N-Glycans with tetraantennary+1 repeat structure;
(f) an erythropoiesis-stimulating glycoprotein having N-Glycans with tetraantennary+2 repeats structure;
(g) an erythropoiesis-stimulating glycoprotein having 14 sialic acid residues (Isoform 2);
(h) an erythropoiesis-stimulating glycoprotein having 13 sialic acid residues (Isoform 3);
(i) an erythropoiesis-stimulating glycoprotein having 12 sialic acid residues (Isoform 4);
(j) an erythropoiesis-stimulating glycoprotein having 11 sialic acid residues (Isoform 5);
(k) an erythropoiesis-stimulating glycoprotein having 10 sialic acid residues (Isoform 6);
(l) an erythropoiesis-stimulating glycoprotein having 9 sialic acid residues (Isoform 7); and
(m) an erythropoiesis-stimulating glycoprotein having 8 sialic acid residues (Isoform 8).
37. The use of any one of embodiments 34 to 36, wherein in the erythropoietic composition
(a) about 3.3 area-% to about 3.8 area-% of the N-Glycans of the erythropoiesis-stimulating glycoprotein have biantennary structure;
(b) about 8.6 area-% to about 9.5 area-% of the N-Glycans of the erythropoiesis-stimulating glycoprotein have triantennary structure;
(c) about 5.6 area-% to about 5.9 area-% of the N-Glycans of the erythropoiesis-stimulating glycoprotein have triantennary+1 repeat structure;
(d) about 42.2 area-% to about 43.4 area-% of the N-Glycans of the erythropoiesis-stimulating glycoprotein have tetraantennary structure;
(e) about 27.4 area-% to about 28.1 area-% of the N-Glycans h of the erythropoiesis-stimulating glycoprotein have a tetraantennary+1 repeat structure;
(f) about 10.7 area-% to about 11.6 area-% of the N-Glycans of the erythropoiesis-stimulating glycoprotein have a tetraantennary+2 repeat structure,
(g) about 13.2 area-% to about 16.0 area-% of the erythropoiesis-stimulating glycoprotein are of Isoform 2;
(h) about 24.1 area-% to about 26.5 area-% of the erythropoiesis-stimulating glycoprotein are of Isoform 3;
(i) about 23.5 area-% to about 24.6 area-% of the N-Glycans of the erythropoiesis-stimulating glycoprotein are of Isoform 4;
(i) about 17.1 area-% to about 18.6 area-% of the N-Glycans of the erythropoiesis-stimulating glycoprotein are of Isoform 5;
(k) about 9.4 area-% to about 11.8 area-% of the N-Glycans of the erythropoiesis-stimulating glycoprotein are of Isoform 6;
(l) about 3.7 area-% to about 5.5 area-% of the erythropoiesis-stimulating glycoprotein are of Isoform 7; and/or
(m) about 0.9 area-% to about 1.6 area-% of the erythropoiesis-stimulating glycoprotein are of Isoform 8,
preferably wherein (a) to (f) are determined by anion exchange chromatography after enzymatic release of N-glycosidically-linked oligosaccharides from the glycoprotein and desialylation and (g) to (m) are determined by capillary zone electrophoresis.
38. The use of any one of embodiments 34 to 37, wherein in the erythropoietic composition
(a) about 42.2 area-% to about 43.4 area-% of the N-Glycans of the erythropoiesis-stimulating glycoprotein have tetraantennary structure;
(b) about 27.4 area-% to about 28.1 area-% of the N-Glycans of the erythropoiesis-stimulating glycoprotein have a tetraantennary+1 repeat structure;
(c) about 10.7 area-% to about 11.6 area-% of the N-Glycans of the erythropoiesis-stimulating glycoprotein have a tetraantennary+2 repeat structure;
(d) about 13.2 area-% to about 16.0 area-% of the erythropoiesis-stimulating glycoprotein are of Isoform 2; and/or
(e) about 24.1 area-% to about 26.5 area-% of the erythropoiesis-stimulating glycoprotein are of Isoform 3,
preferably wherein (a) to (c) are determined by anion exchange chromatography after enzymatic release of N-glycosidically-linked oligosaccharides from the glycoprotein and desialylation and (d) to (e) are determined by capillary zone electrophoresis.
39. The use of any one of embodiments 34 to 38, wherein in the erythropoietic composition
(a) about 42.2 area-% to about 43.4 area-% of the N-Glycans of the erythropoiesis-stimulating glycoprotein have tetraantennary structure;
(b) about 27.4 area-% to about 28.1 area-% of the N-Glycans of the erythropoiesis-stimulating glycoprotein have a tetraantennary+1 repeat structure; and/or
(c) about 10.7 area-% to about 11.6 area-% of the N-Glycans of the erythropoiesis-stimulating glycoprotein have a tetraantennary+2 repeat structure preferably as determined by anion exchange chromatography after enzymatic release of N-glycosidically-linked oligosaccharides from the glycoprotein and desialylation.
40. The use of any one of embodiments 34 to 39, wherein about 27.4 area-% to about 28.1 area-% of the N-Glycans of the erythropoiesis-stimulating glycoprotein in the erythropoietic composition have a tetraantennary+1 repeat structure preferably as determined by anion exchange chromatography after enzymatic release of N-glycosidically-linked oligosaccharides from the glycoprotein and desialylation.
41. The use of any one of embodiments 34 to 40, wherein the erythropoietic composition has an average sialic acid content from about 10.5 to 11.0 moles of sialic acid per mole of the erythropoiesis-stimulating polypeptide.
42. The use of any one of embodiments 34 to 41, wherein the erythropoiesis-stimulating glycoprotein is erythropoietin.
43. An erythropoietic composition comprising at least one erythropoiesis-stimulating glycoprotein wherein
(a) about 3.3 area-% to about 3.8 area-% of the N-Glycans of the erythropoiesis-stimulating glycoprotein have biantennary structure;
(b) about 8.6 area-% to about 9.5 area-% of the N-Glycans of the erythropoiesis-stimulating glycoprotein have triantennary structure;
(c) about 5.6 area-% to about 5.9 area-% of the N-Glycans of the erythropoiesis-stimulating glycoprotein have triantennary+1 repeat structure;
(d) about 42.2 area-% to about 43.4 area-% of the N-Glycans of the erythropoiesis-stimulating glycoprotein have tetraantennary structure;
(e) about 27.4 area-% to about 28.1 area-% of the N-Glycans of the erythropoiesis-stimulating glycoprotein have a tetraantennary+1 repeat structure;
(f) about 10.7 area-% to about 11.6 area-% of the N-Glycans of the erythropoiesis-stimulating glycoprotein have a tetraantennary+2 repeat structure;
(g) about 13.2 area-% to about 16.0 area-% of the erythropoiesis-stimulating glycoprotein are of Isoform 2;
(h) about 24.1 area-% to about 26.5 area-% of the erythropoiesis-stimulating glycoprotein are of Isoform 3;
(i) about 23.5 area-% to about 24.6 area-% of the N-Glycans of the erythropoiesis-stimulating glycoprotein are of Isoform 4;
(j) about 17.1 area-% to about 18.6 area-% of the N-Glycans of the erythropoiesis-stimulating glycoprotein are of Isoform 5;
(k) about 9.4 area-% to about 11.8 area-% of the N-Glycans of the erythropoiesis-stimulating glycoprotein are of Isoform 6;
(l) about 3.7 area-% to about 5.5 area-% of the erythropoiesis-stimulating glycoprotein are of Isoform 7; and/or
(m) about 0.9 area-% to about 1.6 area-% of the erythropoiesis-stimulating glycoprotein are of Isoform 8,
preferably wherein (a) to (f) are determined by anion exchange chromatography after enzymatic release of N-glycosidically-linked oligosaccharides from the glycoprotein and desialylation and (g) to (m) are determined by capillary zone electrophoresis.
44. The erythropoietic composition of embodiment 43, wherein:
(a) about 42.2 area-% to about 43.4 area-% of the N-Glycans of the erythropoiesis-stimulating glycoprotein have tetraantennary structure;
(b) about 27.4 area-% to about 28.1 area-% of the N-Glycans of the erythropoiesis-stimulating glycoprotein have a tetraantennary+1 repeat structure;
(c) about 10.7 area-% to about 11.6 area-% of the N-Glycans of the erythropoiesis-stimulating glycoprotein have a tetraantennary+2 repeat structure;
(d) about 13.2 area-% to about 16.0 area-% of the erythropoiesis-stimulating glycoprotein are of Isoform 2; and/or about 24.1 area-% to about 26.5 area-% of the erythropoiesis-stimulating glycoprotein are of Isoform 3,
preferably wherein (a) to (c) are determined by anion exchange chromatography after enzymatic release of N-glycosidically-linked oligosaccharides from the glycoprotein and desialylation and (d) to (e) are determined by capillary zone electrophoresis.
45. The erythropoietic composition of embodiment 43 or 44, wherein:
(a) about 42.2 area-% to about 43.4 area-% of the N-Glycans of the erythropoiesis-stimulating glycoprotein have tetraantennary structure;
(b) about 27.4 area-% to about 28.1 area-% of the N-Glycans of the erythropoiesis-stimulating glycoprotein have a tetraantennary+1 repeat structure; and/or
(c) about 10.7 area-% to about 11.6 area-% of the N-Glycans of the erythropoiesis-stimulating glycoprotein have a tetraantennary+2 repeat structure, preferably as determined by anion exchange chromatography after enzymatic release of N-glycosidically-linked oligosaccharides from the glycoprotein and desialylation.
46. The erythropoietic composition of any one of embodiments 43 to 45, wherein about 27.4 area-% to about 28.1 area-% of the N-Glycans of the erythropoiesis-stimulating glycoprotein have a tetraantennary+1 repeat structure, preferably as determined by anion exchange chromatography after enzymatic release of N-glycosidically-linked oligosaccharides from the glycoprotein and desialylation.
47. The erythropoietic composition of any one of embodiments 43 to 46, wherein the erythropoietic composition has an average sialic acid content from about 10.5 to 11.0 moles of sialic acid per mole of the erythropoiesis-stimulating glycoprotein.
48. The erythropoietic composition of any one of embodiments 43 to 47, wherein the erythropoietic composition has been produced by a method of any one of embodiments 1 to 20.
49. A glycoprotein, in particular an erythropoiesis-stimulating glycoprotein, particularly erythropoietin, obtained by a method according to any one of embodiments 1 to 29.
50. Pharmaceutical composition containing a glycoprotein produced with a method of any one of embodiments 1 to 29 or an erythropoietin according to any one of embodiments 43 to 48 together with a pharmaceutical diluent, auxiliary agent and/or carrier agent.

### EXAMPLES

The following are examples of methods and compositions of the invention. It is understood that various other embodiments may be practiced, given the general description provided above.

### Example 1—Recombinant Production of Erythropoietin in CHO Cells

Erythropoietin (EPO) was produced in CHO cells in a batch mode. For main stage fermentation, a carbonate-buffered, serum-free cultivation medium was used as the cultivation medium which consisted of the base medium DME (HG) HAM's F-12 modified (R5) (GRH Biosciences/Hazleton Biologics, Denver, USA, Order No. 57-736), sodium hydrogen carbonate, L-(+) glutamine, D-(+) glucose, recombinant insulin, sodium selenite, diaminobutane, hydrocortisone, iron (II) sulfate, asparagine, aspartic acid, serine and polyvinyl alcohol. After the bioreactor was sterilized according to GMP, it was filled with the cultivation medium and sparged with a defined gas mix containing a defined fraction of carbon dioxide. pH control was set inactive during the sparging period. The pH was determined, either by a) taking a sample via a sample port and transferring the sample to an offline analyzer where the sample pH was determined, or by b) measuring the carbon dioxide concentration in the bioreactor exhaust and calculating the pH based on an applicable, predetermined relationship between carbon dioxide concentration and corresponding pH value. The bioreactor pH probe signals were adjusted (by one point calibration) onto the pH determined either with procedure a) or b). The bioreactor was inoculated with inoculum culture and the pH control loop was set active. During the fermentation phase, pH was controlled, and if necessary, the pH of the culture was adjusted to the set point of pH 6.95 by adding 1 mol/L NaOH or increasing CO₂ influx, respectively. After about 5 days, the fermenter content was harvested. Intact CHO cells and cell fragments were removed from the fermentation supernatant by disk stack separation and discarded. The pH of the filtered cell-free culture supernatant was adjusted with acetic acid (2 mol/L) to pH 5.0-5.2, incubated for several hours and the pH-adjusted solution was subsequently filtered at 1-9° C.

### Example 2—Determination of Media Specific Correlation of pH and Corresponding Exhaust Carbon Dioxide Concentration for EPO Production Process

In order to empirically determine the media specific correlation between pH and the corresponding exhaust carbon dioxide concentration for the fermentation process described in Example 1, the bioreactor was filled with cultivation medium as described therein. The bioreactor pH was determined with a built-in online-pH probe that was initially two-point calibrated in buffers (4.00 and 7.00 at 25° C., Mettler Toledo). Carbon dioxide gas was used as an acidic pH correction agent via pH control to maintain pH at upper dead bands. The pH controller was set up as a proportional controller to achieve a constant carbon dioxide influx. No alkaline correction agent was used, and so pH thereby naturally increases by carbon dioxide removal via constant aeration with process air until pH controller adds carbon dioxide to maintain pH at upper dead band. After sterilization and media fill a hold step was defined to stabilize pH, pressure and temperature. To enable accurate pH readings, a port in the bioreactor lid was then opened, making the system unsterile in the process. Two independent pH-probes connected to respective pH-meters were inserted through the open port into the bioreactor to measure pH in the liquid phase without sampling. Agitation, aeration and temperature control all stayed active. Pressure control was set to inactive. pH-meters were independently three point calibrated (calibration buffers at 9.21, 7.00, 4.00 at 25° C.) with active automated temperature compensation (ATC). The average of the pH-meter readings was used to adjust (via one point calibration) the online bioreactor pH probe signal. Maximum acceptable difference of the pH meter signals was 0.02 units, and the maximum acceptable difference from the buffer pH was 0.01 units. The same procedure was performed again to check bioreactor pH after the experiment to detect any unintended drifts of the bioreactor probe signal. After standardization of the bioreactor probe signal, the bioreactor lid was closed again and pressure control was set as active. After establishing an equilibrium (stable pH and carbon dioxide concentration in the exhaust), the first data point online pH and corresponding exhaust carbon dioxide concentration was documented. Correlations were determined in at least four technical replicates, which means at least four independent bioreactors filled with identical medium, and fitted with quadratic regression to result in a final equation of the form

$$pH = a - b * [ {CO}_{2} ] + c * {[ {CO}_{2} ]}^{2}$$

Four pH set points were determined in each bioreactor. After establishing an equilibrium at every set point, pH was also determined via a sample-based offline measurement.

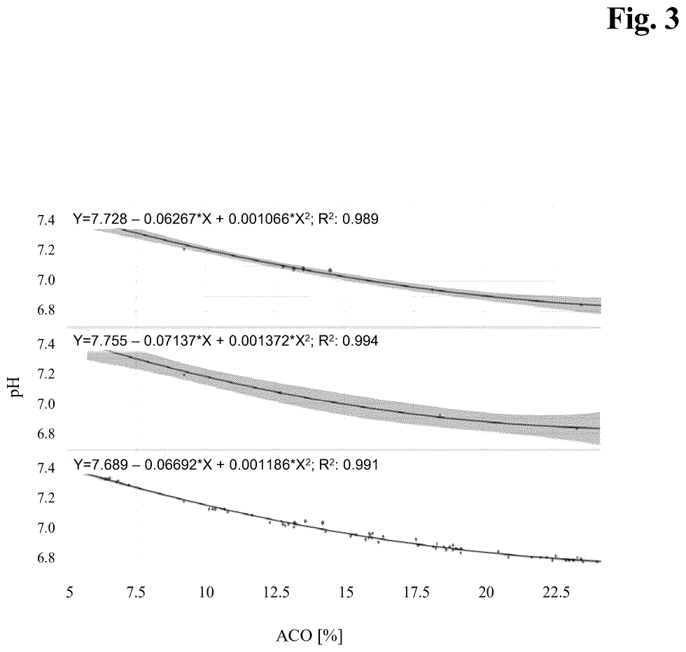

The media specific correlation of the production medium lots is shown in FIG. 3 for three pressures, 20 mbar (top), 50 mbar (middle) and 135 mbar (bottom). Additionally, some data from 10 L scale is depicted that has been used to prove scale independency of the pH determined (dots from small scale, diamonds from 10 L stainless steel bioreactors).

Additionally, the offset between sample-based offline pH values in manufacturing scale and the pH in the bioreactor described by the relationship as shown in FIG. 3 has been determined to be 0.05 pH units in an at-scale validation where the bioreactor pH, determined via exhaust carbon dioxide concentration, was compared to sample based offline pH. An average difference in pH of 0.05 units has been determined in all scales. In order to account for the offset determined, the pH set point of the erythropoietin fermentation has been decreased from pH 7.00+/−0.05 to 6.95+/−0.05. The final equation to calculate pH based on the exhaust carbon dioxide concentration therefore yields the true pH, that means the pH without any offsets that may be introduced by sample based offline measurement.

The quality of the model, i.e. the ability of the model to describe the data points modeled may, as one example, be described by the regression coefficient R-squared (R²). An R² of 1 thereby means that all data points are perfectly described by the model. In all models R-squared was larger than 0.98. The root mean squared error was 0.02 in all models. Taking into account potential errors of calibration, environmental factors and the like the accuracy of the present method is considered to be +/−0.03 pH units.

### Example 3—Blue Sepharose Chromatography

Erythropoietin polypeptide was purified essentially as described in WO 2010/34442. A chromatography column (Amicon P440×500, Amicon, GB) was filled with about 500 L Blue Sepharose and regenerated with 0.5 N NaOH. Subsequently the column was equilibrated with about three column volumes (CV) equilibration buffer (20 mM Na acetate, 5 mM CaCl₂), 0.1 M NaCl, pH 5.0±0.2). Erythropoietin binds to this support at low ionic strength and neutral to acidic pH values. The cell-free culture supernatant from Example 1 was absorbed to the column and the column was rewashed with about 1 CV wash buffer 1 (20 mM Na acetate, 5 mM CaCl₂), 0.25 M NaCl, pH 5.0±0.2) followed by about 2 CV wash buffer 2 (20 mM TRIS-HCl, 5 mM CaCl₂, pH 6.9±0.2). Subsequently erythropoietin was eluted with about 2 CV elution buffer (100 mM TRIS-HCl, 5 mM CaCl₂, 1 M NaCl, pH 9.0±0.2), by increasing the ionic strength and the pH value. The entire protein peak was collected, adjusted to pH 6.9 with HCl and stored until further processing.

### Example 4—Butyl Toyopearl Chromatography (Hydrophobic Chromatography)

A chromatography column (Pharmacia BPG 300/500) was filled with about 200 l Butyl Toyopearl (Tosoh Haas) and regenerated with 4.6 M guanidine-HCl and 0.5 N NaOH. Subsequently the column was equilibrated with at least three CV equilibration buffer (20 mM TRIS-HCl, 5 mM CaCl₂, 0.75 M NaCl, 10% 2-propanol, pH 6.9±0.2). The eluate of the Blue Sepharose column of Example 3 was adjusted to 10% 2-propanol and absorbed to the column. The column was rewashed with about one CV equilibration buffer and then with about two CV wash buffer (20 mM TRIS-HCl, 5 mM CaCl₂, 0.75 M NaCl, 19% 2-propanol, pH 6.9+0.2). Subsequently the erythropoietin was eluted with about two CV elution buffer (20 mM TRIS-HCl, 5 mM CaCl₂, 0.75 M NaCl, 27% 2-propanol, pH 6.9±0.2). The entire protein peak was collected, immediately diluted three-fold with dilution buffer (20 mM TRIS-HCl, 5 mM CaCl₂, pH 6.9±0.2) and stored until further processing.

### Example 5—Hydroxyapatite Ultrogel Chromatography

The chromatography column (Sartorius 100×50 cm, Sartorius GB) was packed with about 200 L hydroxyapatite Ultrogel and regenerated with regeneration buffer 1 (0.2 M potassium phosphate, 0.1 mM CaCl₂), pH 6.9±0.2) and subsequently with 0.5 N NaOH. Subsequently the column was equilibrated with about 3 CV equilibration buffer (20 mM Tris-HCl, 5 mM CaCl₂, 0.25 M NaCl, 9% Isopropanol, pH 6.9±0.2). The eluate from Example 4 was adsorbed to the column. The column was rewashed with about one CV equilibration buffer and then with about two CV wash buffer (10 mM Tris-HCl, 5 mM CaCl₂, pH 6.9±0.2). It was subsequently eluted with about 2.5 CV elution buffer (10 mM Tris-HCl, 0.5 mM CaCl₂, 10 mM potassium phosphate pH 6.9±0.2). The entire protein peak was collected and stored until further processing.

### Example 6—Reversed Phase HPLC (RP-HPLC)

The eluate from Example 5 was filtrated with a nanofilter to remove any viruses. The preparative HPLC was carried out with a Merck Prepbar 100 separation system (or equivalent). The separation column (30×50 cm) was packed with Vydac C4 material (Grace, USA). Before use the column was regenerated by applying several times a gradient of buffer A (0.1% trifluoroacetic acid in water) to 100% solvent and was subsequently equilibrated with buffer A. The eluate of the hydroxyapatite column was acidified to about pH 2.5 with trifluoroacetic acid and sterilized by filtration. Subsequently the erythropoietin was absorbed to the column at a temperature of 22±4° C. and a flow rate of 2700 ml/min. The column was eluted with a linear gradient of buffer A to buffer B (80% acetonitrile, 0.1% trifluoroacetic acid in water) at the same temperature and flow rate. The elution peak was collected in fractions. The eluate was immediately neutralized by adding seven volumes HPLC dilution buffer (10 mM Na/K phosphate, pH 7.5±0.2). Fractions which had a purity of at least 99% in the analytical HPLC were pooled (pool volume about 60 l).

### Example 7—DEAE Sepharose FF Chromatography

A chromatography column (Merck Quickscale 25×50 cm) was filled with 7.5 L DEAE Sepharose Fast Flow (Cytiva) gel per g of erythropoietin in the applied sample and regenerated with 1 M NaOH. Subsequently the column was equilibrated with 100 mM NaKPO₄ pH 7.5±0.2 and afterwards with at least 10 CV 10 mM sodium/potassium phosphate, pH 7.5+0.2. The eluate of the HPLC column from Example 6 was absorbed to the column, and the column was washed with at least five CV equilibration buffer and then with about ten CV wash buffer (30 mM Na acetate, pH 4.5±0.1). Subsequently the column was again washed with about ten CV equilibration buffer and the erythropoietin was eluted with about four CV elution buffer (10 mM sodium/potassium phosphate, 80 mM NaCl, pH 7.5±0.2). The entire protein peak was collected, the conductivity and the pH of the DEAE eluate were adjusted, sterilized by filtration, dispensed and stored.

### Example 8—Analytical Methods Used for Product Characterization

### Sugar Analysis

For determination of the relative content of the N-Glycans an enzymatic test procedure was used. In this procedure the N-glycosidically-linked oligosaccharides of erythropoietin were cleaved by the enzyme N-glycosidase F. In addition, the terminal sialic acids were removed from the oligosaccharides with the aid of the enzyme neuraminidase. After removal of the protein fraction via ultrafiltration, the N-oligosaccharides that were obtained were separated and analyzed using a High-Performance Anion-Exchange Chromatography system with Pulsed Amperometric Detection (HPAEC-PAD) and a suitable data recording system. An erythropoietin reference standard which was treated in exactly the same manner as the samples was measured during the series of tests. The area-percentage of each glycosylated variant in the preparation was calculated from the corresponding peak in the chromatogram of the glycans produced (e.g. biantennary, triantennary, triantennary+1 repeat, tetraantennary, tetraantennary+1 repeat, tetraantennary+2 repeats).

### Determination of the Isoform Distribution

The relative content of isoforms was determined by means of capillary zone electrophoresis. In this method, separation is carried out in a constant field strength in an uncoated glass capillary with a uniform buffer system, the pH of which lies above the pI-values of the EPO isoforms. Since all EPO isoforms are negatively charged and are transported by the endoosmotic flow towards the cathode, the basic isoforms are detected first. For the analysis, the samples were diafiltered in water. The capillary was rinsed with an electrolyte solution followed by the application of the diluted samples into the capillary. The separation took place by applying a high voltage of 25,000 V. The mobile buffer had an excess of positive ions which resulted in an electroosmotic flow. Use of a quartz capillary enabled a photometric detection of the proteins in the capillary using a diode array detector and a quantitative determination by integrating the peaks corresponding to Isoform 1 to Isoform 9 to obtain the area under the peak [relative area-%].

### Determination of the Content of Sialic Acid Residues

The sialic acid content was determined chromatographically by means of HPAEC-PAD after enzymatic cleavage of the sialic acids with neuraminidase. For this, erythropoietin samples were diluted in a 5 mM Na phosphate buffer, pH 7.2. Half of each preparation was used to determine the EPO amount by means of RP-HPLC. Neuraminidase was added to the second half of the preparations and incubated overnight at 37° C. Subsequently, the digestion mixtures were split in half, diluted with water and 50 μl thereof were applied to the HPAEC-PAD. The amount of sialic acids in the applied sample was determined with the help of a calibration line which was obtained from values of a sialic acid standard that was also analysed. The sialic acid content (mole sialic acid/mole EPO) was calculated from the result of the sialic acid determination and the determination of the amount of EPO used by means of RP-HPLC.

### Exclusion Chromatography (SE-HPLC)

For SE-HPLC the chromatography column was equilibrated with three to five column volumes (CV) buffer to obtain a uniform baseline without additional peaks before the separation was started. The samples were diluted to a concentration of 0.2 mg/mL and injected into the SE-HPLC. The peaks were integrated according to standard methods. The peaks were separated from one another by dropping perpendiculars.

### Peptide Map

Incompletely glycosylated erythropoietin species such as de-O-EPO and de-N-EPO can be quantitatively determined by peptide mapping. For this purpose the erythropoietin molecule was cleaved into peptides by the endoproteinase Lys-C and these peptides were separated by HPLC. The peptide pattern obtained was compared with a reference standard. The results obtained were compared with the standard with regard to peak size, peak appearance and retention time.

### Example 9—Effects of pH Determination on Product Quality

To compare the effects of the different methods for pH calibration and determination, ten batches of erythropoietin were produced as described in the examples 1-8, using the pH determination method according to the invention. For standardization of the bioreactor pH-probe signal, the cultivation medium that had been filled into the bioreactor after sterilization was subsequently equilibrated with a defined gas mixture (93% process air and 7% CO₂) until saturation. After the equilibration phase, the pH in the cultivation medium was calculated from the exhaust carbon dioxide concentration based on the media specific correlation determined according to the method described in Example 2. Bioreactor probe signal was then adjusted onto the calculated pH value.

As a control, ten batches were used that had been produced according to the same manufacturing process, but where calibrating the pH probe in the fermenter had been performed off-line according to standard methods known in the art. For this, after sterilization of the fermenter, the cultivation medium was added to the fermenter, a sample was removed and the sample pH was determined offline using a pH meter that was 3-point calibrated as described in Example 2. The in-line pH probe was then adjusted to the pH value measured offline. One of the control runs had to be terminated and harvested pre-maturely and was thus excluded for the yield analysis.

TABLE 2
Erythropoietin product yield

| | pH determination via | | | | | |
|---|---|---|---|---|---|---|
| | Sample based method | | | CO₂-based method | | |
| | Me- | Standard | | Me- | Standard | |
| Parameter | dian | deviation | Range | dian | deviation | Range |
| Product | 136 | 17.8 | 107-160 | 142 | 10.3 | 131-168 |
| amount | | | | | | |
| [g] | | | | | | |

As can be seen from Table 2, the variability of the product yield is reduced in runs where the in-line pH probe has been adjusted using the carbon dioxide-based calibration method. The standard deviation of the nine control runs that have been adjusted to sample based offline pH is much higher compared to the ten runs using the carbon dioxide based method.

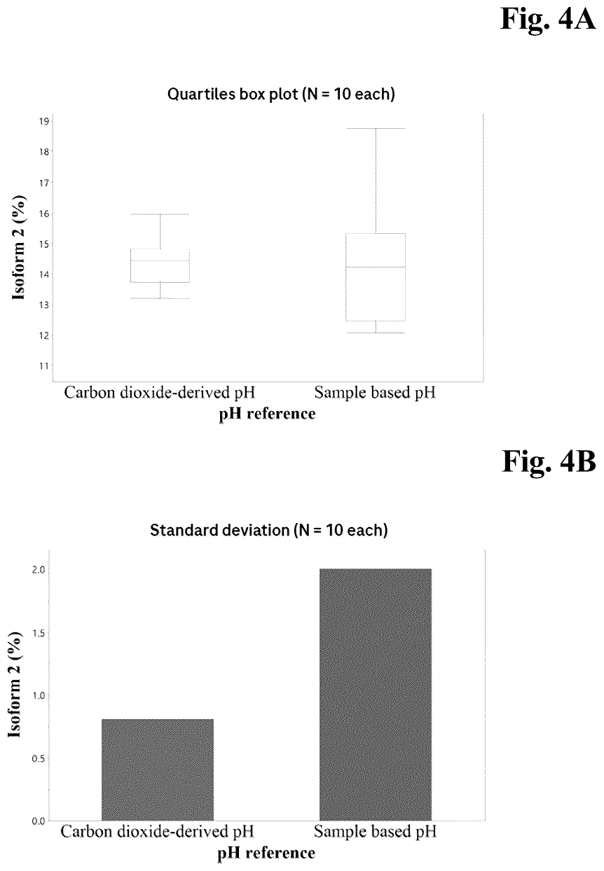

Results of the determination of sialylated Isoforms 2-8 using capillary zone electrophoresis (analytical method as described in example 8) are shown in Table 3. FIG. 4 shows an exemplary visual representation of the results for Isoform 2. The ranges between measured minimum and maximum values and accordingly also the standard deviation are considerably reduced for many parameters.

TABLE 3
Erythropoietin Sialylation

| | pH determination via | | | | | |
|---|---|---|---|---|---|---|
| | Sample based method | | | CO₂-based method | | |
| Parameter | Me- | Standard | | Me- | Standard | |
| [area-%] | dian | deviation | Range | dian | deviation | Range |
| Isoform 2 | 14.2 | 2.0 | 12.1-18.7 | 14.4 | 0.8 | 13.2-16.0 |
| Isoform 3 | 25.1 | 1.7 | 23.0-29.0 | 25.1 | 0.7 | 24.1-26.5 |
| Isoform 4 | 23.9 | 0.5 | 23.3-24.9 | 24.1 | 0.4 | 23.5-24.6 |
| Isoform 5 | 17.9 | 0.9 | 15.7-18.9 | 17.8 | 0.5 | 17.1-18.6 |
| Isoform 6 | 10.9 | 1.4 | 7.7-12.4 | 11.0 | 0.6 | 9.4-11.8 |
| Isoform 7 | 5.0 | 1.2 | 2.7-6.7 | 5.0 | 0.5 | 3.7-5.5 |
| Isoform 8 | 1.3 | 0.5 | 0.7-2.3 | 1.4 | 0.2 | 0.9-1.6 |

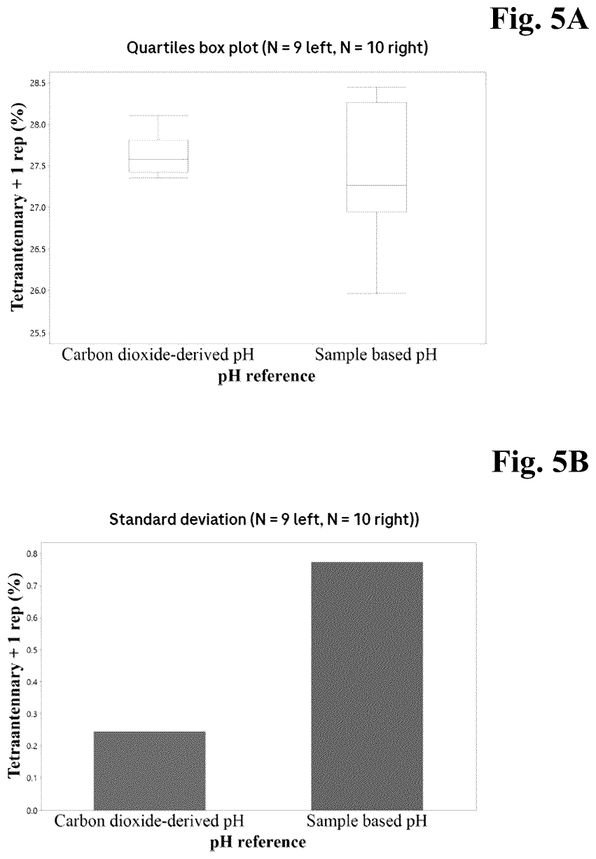

Results of the determination of desialylated N-Glycans using anion exchange chromatography after enzymatic release of N-glycosidically-linked oligosaccharides from the glycoprotein and desialylation (analytical method as described in example 8) are shown in Table 4. FIG. 5 shows an exemplary visual representation of the results for N-glycans with tetraantennary+1 repeat. As with the sialylated isoforms, the ranges between measured minimum and maximum values and accordingly also the standard deviation are considerably reduced for many parameters.

TABLE 4
Erythropoietin Glycosylation

| | pH determination via | | | | | |
|---|---|---|---|---|---|---|
| | Sample based method | | | CO₂-based method | | |
| | | Standard- | | | Standard | |
| Parameter | Median | deviation | Range | Median | deviation | Range |
| Biantennary | 3.3 | 0.2 | 3.0-3.5 | 3.5 | 0.2 | 3.3-3.8 |
| (area-%) | | | | | | |
| Triantennary | 9.2 | 0.4 | 8.6-9.8 | 9.3 | 0.3 | 8.6-9.5 |
| (area-%) | | | | | | |
| Triantennary + | 5.8 | 0.2 | 5.4-6.1 | 5.7 | 0.1 | 5.6-5.9 |
| 1 repeat | | | | | | |
| (area-%) | | | | | | |
| Tetraantennary | 43.8 | 1.4 | 41.6-46.3 | 43.0 | 0.4 | 42.2-43.4 |
| (area-%) | | | | | | |
| Tetraantennary + | 27.3 | 0.8 | 26.0-28.4 | 27.6 | 0.2 | 27.4-28.1 |
| 1 repeat | | | | | | |
| (area-%) | | | | | | |
| Tetraantennary + | 10.4 | 0.9 | 9.4-11.9 | 11.0 | 0.3 | 10.7-11.6 |
| 2 repeats | | | | | | |
| (area-%) | | | | | | |
| Sialic acid ratio | 11.0 | 0.3 | 10.6-11.7 | 10.9 | 0.2 | 10.5-11.0 |
| (mol/mol) | | | | | | |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for recombinantly producing a glycoprotein composition comprising at least one glycoprotein, wherein the method comprises the step of
cultivating a recombinant host cell which expresses the glycoprotein in a cultivation medium in a bioreactor having a pH measuring device positioned in the bioreactor and configured to be in physical contact with the cultivation medium,
characterized in that
(a) the cultivating is performed under sterile conditions,
(b) the pH value measured with the pH measuring device differs by 0.05 units or less from the pH value of the cultivation medium, and
(c) the relative content of at least one glycosylated variant in the glycoprotein composition has reduced batch-to-batch variability, compared to a method wherein the pH value measured by the pH measuring device differs by more than 0.03 units, or by more than 0.05 units, from the pH value in the cultivation medium,
and thereby producing the glycoprotein composition.

2. A method for recombinantly producing a glycoprotein composition comprising at least one glycoprotein in a recombinant host cell which expresses the glycoprotein, comprising the steps of
(a) providing a bioreactor comprising a pH measuring device positioned in the bioreactor and configured to be in physical contact with a cultivation medium,
(b) closing and sterilizing the bioreactor,
(c) filling the cultivation medium into the bioreactor,
(d) calibrating the pH measuring device,
(e) inoculating the bioreactor with the recombinant host cell,
(f) cultivating the recombinant host cell under conditions suitable for producing the glycoprotein composition, and
(g) thereby producing the glycoprotein composition,
characterized in that the calibrating of step (d) comprises the steps of
(i) introducing a gas mixture comprising carbon dioxide gas into the bioreactor,
(ii) determining the carbon dioxide concentration in the headspace and/or the exhaust,
(iii) calculating the pH value of the cultivation medium based on a media specific correlation, and
(iv) adjusting the pH measuring device to the pH value calculated in step (iii).

3. The method of claim 2, characterized in that the pH value measured with the pH measuring device after the calibration step differs by 0.05 units or less, or by 0.03 units or less, from the pH value of the cultivation medium.

4. The method of claim 1 or 2, characterized in that the glycoprotein composition comprises at least one glycosylated variant of the glycoprotein, and
that the standard deviation for the relative content of the glycosylated variant calculated for glycoprotein compositions from at least two fermentation batches is decreased, compared to the standard deviation of the relative content of the glycosylated variant calculated for glycoprotein compositions which have been produced using a method where the pH value measured by the pH measuring device differs by more than 0.05 units, preferably by more than 0.03 units, from the pH value in the cultivation medium.

5. The method of claim 1 or 2, wherein the recombinant host cell is a mammalian cell, preferably a CHO cell.

6. The method of claim 4, wherein the glycosylated variant is selected from the group consisting of N-glycans variants, O-glycan variants, sialylation variants, mannosylation variants, galactosylation variants and fucosylation variants.

7. The method of claim 1 or 2, wherein the cultivation medium comprises a carbonate buffer system.

8. The method of claim 1, comprising the step of maintaining the pH in the cultivation medium at a desired set point.

9. The method of claim 1 or 2, wherein the glycoprotein composition is an erythropoietic composition and the glycoprotein is an erythropoiesis-stimulating glycoprotein.

10. The method of claim 9, further characterized in that the erythropoietic composition comprises a defined amount of at least one glycosylated variant of an erythropoiesis-stimulating glycoprotein selected from the group consisting of
(a) an erythropoiesis-stimulating glycoprotein having N-Glycans with biantennary structure,
(b) an erythropoiesis-stimulating glycoprotein having N-Glycans with triantennary structure,
(c) an erythropoiesis-stimulating glycoprotein having N-Glycans with triantennary+1 repeat structure,
(d) an erythropoiesis-stimulating glycoprotein having N-Glycans with tetraantennary structure,
(e) an erythropoiesis-stimulating glycoprotein having N-Glycans with tetraantennary+1 repeat structure,
(f) an erythropoiesis-stimulating glycoprotein having N-Glycans with tetraantennary+2 repeats structure,
(g) an erythropoiesis-stimulating glycoprotein having 14 sialic acid residues (Isoform 2),
(h) an erythropoiesis-stimulating glycoprotein having 13 sialic acid residues (Isoform 3),
(i) an erythropoiesis-stimulating glycoprotein having 12 sialic acid residues (Isoform 4),
(j) an erythropoiesis-stimulating glycoprotein having 11 sialic acid residues (Isoform 5),
(k) an erythropoiesis-stimulating glycoprotein having 10 sialic acid residues (Isoform 6),
(l) an erythropoiesis-stimulating glycoprotein having 9 sialic acid residues (Isoform 7), and
(m) an erythropoiesis-stimulating glycoprotein having 8 sialic acid residues (Isoform 8),
wherein the standard deviation for the relative content of the glycosylated variant calculated for erythropoietic compositions from at least two fermentation batches is decreased, compared to the standard deviation of the relative content of the glycosylated variant calculated for erythropoietic compositions which have been produced using a method where the measured pH value differs by more than 0.03 units, or by more than 0.05 units, from the pH value in the cultivation medium.

11. The method of claim 9, characterized in that the erythropoietic composition comprises one or more selected from the group consisting of:
(a) about 3.3 area-% to about 3.8 area-% erythropoiesis-stimulating glycoprotein having N-Glycans with biantennary structure;
(b) about 8.6 area-% to about 9.5 area-% erythropoiesis-stimulating glycoprotein having N-Glycans with triantennary structure;
(c) about 5.6 area-% to about 5.9 area-% erythropoiesis-stimulating glycoprotein having N-Glycans with triantennary+1 repeat structure;
(d) about 42.2 area-% to about 43.4 area-% erythropoiesis-stimulating glycoprotein having N-Glycans with tetraantennary structure;
(e) about 27.4 area-% to about 28.1 area-% erythropoiesis-stimulating glycoprotein having N-Glycans with tetraantennary+1 repeat structure;
(f) about 10.7 area-% to about 11.6 area-% erythropoiesis-stimulating glycoprotein having N-Glycans with tetraantennary+2 repeat structure,
(g) about 13.2 area-% to about 16.0 area-% erythropoiesis-stimulating glycoprotein of Isoform 2;
(h) about 24.1 area-% to about 26.5 area-% erythropoiesis-stimulating glycoprotein of Isoform 3;
(i) about 23.5 area-% to about 24.6 area-% erythropoiesis-stimulating glycoprotein of Isoform 4;
(j) about 17.1 area-% to about 18.6 area-% erythropoiesis-stimulating glycoprotein of Isoform 5;
(k) about 9.4 area-% to about 11.8 area-% erythropoiesis-stimulating glycoprotein of Isoform 6;
(l) about 3.7 area-% to about 5.5 area-% erythropoiesis-stimulating glycoprotein of Isoform 7; and
(m) about 0.9 area-% to about 1.6 area-% erythropoiesis-stimulating glycoprotein of Isoform 8.

12. The method of claim 1 or 2, wherein the glycoprotein is erythropoietin.

13. A method of reducing the variability of the relative content of at least one glycosylated variant between batches of a recombinant glycoprotein, the method comprising:
(a) providing a bioreactor comprising a pH measuring device positioned in the bioreactor and configured to be in physical contact with a cultivation medium,
(b) closing and sterilizing the bioreactor,
(c) filling the cultivation medium into the bioreactor,
(d) calibrating the pH measuring device,
(e) inoculating the bioreactor with a recombinant host cell which expresses the glycoprotein, and
(f) cultivating the recombinant host cell under conditions suitable for producing the glycoprotein, and
(g) thereby producing the glycoprotein,
wherein the calibrating of step (d) comprises the steps of
(i) introducing a gas mixture comprising carbon dioxide gas into the bioreactor,
(ii) determining the carbon dioxide concentration in the headspace and/or the exhaust gas of the bioreactor,
(iii) calculating the pH value of the cultivation medium based on a media specific correlation, and
(iv) adjusting said pH measuring device to the pH value calculated in step (iii), and
wherein steps (a) to (g) result in a first batch of said recombinant glycoprotein with a defined relative content of the glycosylated variant;
(h) repeating the steps (a) to (g) resulting in at least one subsequent batch of said glycoprotein, wherein the relative content of the at least one glycosylated variant of said first and at least one subsequent batch has reduced batch-to-batch variability.

14. Use of the method of claim 2 for reducing the standard deviation of the relative content of at least one glycosylated variant in a glycoprotein composition calculated between at least two fermentation batches.

15. The use of claim 14, wherein the carbon-dioxide based method comprises the steps of
(i) introducing a gas mixture comprising carbon dioxide gas into the bioreactor comprising the cultivation medium,
(ii) determining the carbon dioxide concentration in the headspace and/or the exhaust gas of the bioreactor,
(iii) calculating the pH value of the cultivation medium based on a Media specific correlation, and
(iv) adjusting the pH measuring device to the pH value calculated in step (iii).


SEQUENCE LISTING (1 sequence)

SEQ ID NO: 1 - protein, 165 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGD